(19) United States
(12) Patent Application Publication
(10) Pub. No.: US 2007/0167353 A1
(43) Pub. Date: Jul. 19, 2007

(54) PRODRUG COMPOSITION

(76) Inventors: John Hilfinger, Ann Arbor, MI (US); Wei Shen, Dearborn, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/690,528

(22) Filed: Mar. 23, 2007

Related U.S. Application Data
(63) Continuation-in-part of application No. 10/972,729, filed on Oct. 25, 2004.
(60) Provisional application No. 60/785,582, filed on Mar. 24, 2006.
(60) Provisional application No. 60/514,121, filed on Oct. 24, 2003.

(51) Int. Cl. A61K 38/14; A61K 31/7076; C07H 19/16; C07K 9/00

(52) U.S. Cl. 514/7; 514/8; 530/322; 514/47; 536/27.3

(57) ABSTRACT

A prodrug composition is provided which includes a pharmaceutical species and an amino acid having a covalent bond to the pharmaceutical species. A particular pharmaceutical species is adenosine arabinoside, also known as Ara A and by the trade name vidarabine. Ara A prodrugs of the present invention have increased bioavailability compared to the parent compound Ara A. The inventive prodrug is transported from the gastrointestinal lumen by a specific transporter and is enzymatically cleaved to yield Ara A, such that Ara A is delivered to the individual.

### REFERENCE TO RELATED APPLICATIONS

This application claims priority of U.S. Provisional Patent Application Ser. No. 60/785,582, filed Mar. 24, 2006; and is a continuation-in-part of U.S. patent application Ser. No. 10/972,729, filed Oct. 25, 2004, which claims priority to U.S. Provisional Patent Application Ser. No. 60/514,121, filed Oct. 24, 2003. The entire content of each application is incorporated herein by reference.

### FIELD OF THE INVENTION

The present invention generally relates to prodrugs that are substrates for enzymatic cleavage, and in particular to prodrugs where the enzymatic substrate portion of the prodrug is simultaneously a substrate for a membrane transporter.

### BACKGROUND OF THE INVENTION

A prodrug in vivo activation strategy is considered attractive in increasing the concentration of an active compound at the local site of enzymatic cleavage to an active compound with the concurrent limitation of systemic exposure to the active compound so as to reduce side effects. It is conventional to couple a moiety to an active drug species such that an enzyme associated with the target site acts on the substrate moiety to generate an active species at a desired locality. Enzymes useful in prodrug activation have been described and include enzymes such as thymidine kinase, cytosine deaminase, and purine nucleoside phosphorylase, as described in U.S. Pat. Nos. 5,338,678; 5,552,311; 6,017,896; and 6,027,150. While the basic concept of coupling a substrate moiety to an active species is well known, this approach has met with limited success owing to difficulty in transporting the prodrug into a particular type of cell, and the presence of a cleavage enzyme in cell types other than those targeted for therapeutic interaction with the active drug species. Thus, there exists a need for a prodrug where the enzymatic cleavage substrate bound to the active drug species also serves as a membrane transporter species.

### SUMMARY OF THE INVENTION

A composition is provided according to embodiments of the present invention including a prodrug having the structural formula: 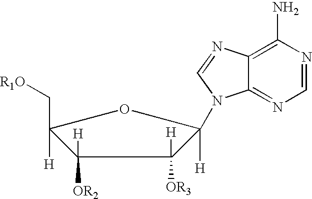
where R₁, R₂ and R₃ are each independently H, or a substrate for a transporter selected from: an amino acid, a dipeptide and a tripeptide, where at least one of R₁, R₂ and R₃ is an amino acid, a dipeptide or a tripeptide. An included amino acid is an L-amino acid and/or a D-amino acid in particular embodiments of an inventive prodrug. An inventive prodrug is optionally provided as a pharmaceutically acceptable salt or hydrate.

Optionally, R₁ is an amino acid, a dipeptide or a tripeptide, and R₂ and R₃ are each independently H, an amino acid, a dipeptide or a tripeptide.

In a further option, R₁ is an amino acid, a dipeptide or a tripeptide, and R₂ and R₃ are both H.

In preferred embodiments, the amino acid, dipeptide or tripeptide substrate for a transporter is a substrate for an intestinal transporter.

In a preferred embodiment of an inventive composition, a prodrug is characterized by at least two-fold greater bioavailability compared to adenine 9-beta-D-arabinofuranoside.

Also provided is a composition including a prodrug having the structural formula: 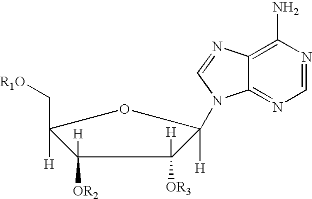
where R₁ is an amino acid and where R₂ and R₃ are both H.

Described herein in specific embodiments of compositions of the present invention are Ara A prodrugs including 5′-O-D-isoleucyl Ara A; 5′-O-L-isoleucyl Ara A; 5′-O-D-valyl Ara A; 5′-O-L-valyl Ara A; 5′-O-glycyl Ara A; 5′-O-D-phenylalanyl Ara A; 5′-O-L-phenylalanyl Ara A; 5′-O-D-leucyl Ara A; 5′-O-L-leucyl Ara A; 5′-O-L-aspartyl Ara A; 5 ′-O-D-alpha-aspartyl Ara A; 5′-O-L-alpha-aspartyl Ara A; 5′-O-D-beta-aspartyl Ara A; 5′-O-L-beta-aspartyl Ara A; and 5′-O-L-prolyl Ara A.

A method of treatment is provided according to embodiments of the present invention which includes administering to a subject in need thereof a therapeutically effective amount of a composition comprising an Ara A prodrug having the structural formula: 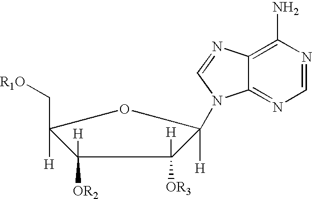
where R₁, R₂ and R₃ are each independently H, an amino acid, a dipeptide or a tripeptide, where at least one of R₁, R₂ and R₃ is an amino acid, a dipeptide or a tripeptide; and a pharmaceutically acceptable carrier.

In particular embodiments, a method of treatment includes administration of an inventive composition to a subject infected or at risk of infection with a virus from a virus family such as Adenoviridae, Arenaviridae, Bunyaviridae, Caliciviridae, Coronaviridae, Flaviviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, and Togaviridae.

In further particular embodiments, a method of treatment includes administration of an inventive composition to a subject having or at risk of having cancer.

A preferred method includes oral administration of an inventive Ara A composition.

Use of a composition according to embodiments of the present invention is described in preparing an antiviral medicament.

### BRIEF DESCRIPTION OF THE DRAWINGS

FIG. 1 is a graph showing floxuridine prodrug analog uptake as mediated by HPEPT1 infected cells as compared to normal cells with 3′ and 5′ valyl esters of floxuridine showing enhanced uptake in HPEPT1/Hela cells with uptake measured for each of the synthesized prodrugs. Cephalexin, a known drug transported by HPEPT1, is included as a positive control;

FIG. 2 is a graph showing activation of floxuridine amino acid prodrug;

FIG. 3A is a graph showing concentration profiles of the disappearance of Ara A and the appearance of Ara H in the presence of adenosine deaminase;

FIG. 3B is a graph showing percent Ara A or prodrug remaining following incubation with adenosine deaminase for 90 minutes;

FIG. 4 is a graph showing mean plasma profiles of Ara A and Ara H after administration of Ara A into the duodenum of a rat;

FIG. 5 is a graph showing mean plasma profiles of Ara A, Ara H, and 5′-O-L Isoleucyl Ara A after administration of 5′-O-L Isoleucyl Ara A into the duodenum of a rat;

FIG. 6 is a graph showing mean plasma profiles of Ara A, Ara H, and 5′-O-D Isoleucyl Ara A after administration of 5′-O-D Isoleucyl Ara A into the duodenum of a rat;

FIG. 7 is a graph showing mean plasma profiles of Ara A, Ara H, and 5′-O-L Valyl Ara A after administration of 5′-O-L Valyl Ara A into the duodenum of a rat;

FIG. 8 is a graph showing mean plasma profiles of Ara A, Ara H, and 5′-O-D Valyl Ara A after administration of 5′-O-D Valyl Ara A into the duodenum of a rat; and

FIG. 9 is a graph showing mean plasma profiles of Ara A, Ara H, and 5′-O-Glycyl Ara A after administration of 5′-O-Glycyl Ara A into the duodenum of a rat.

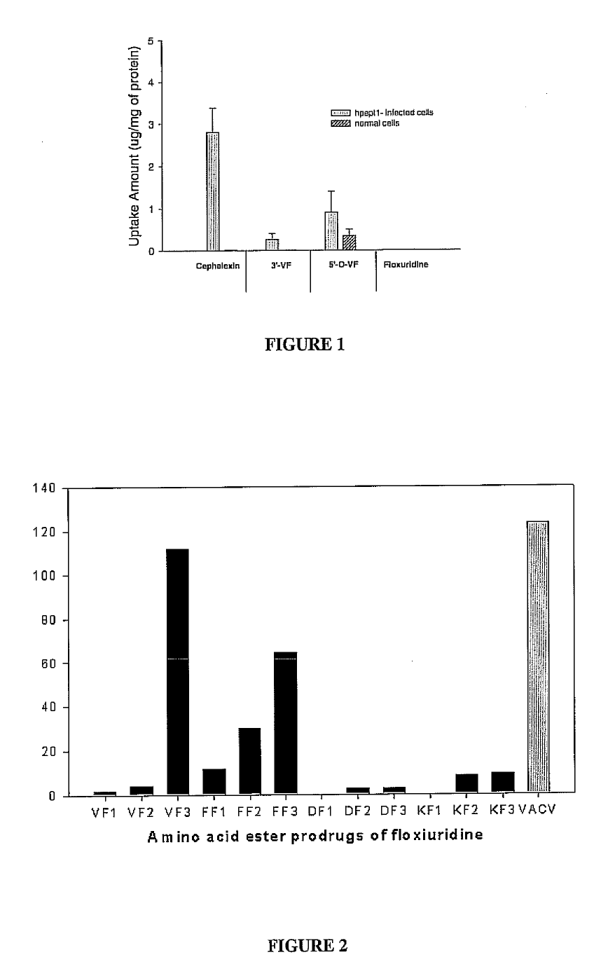

### DETAILED DESCRIPTION OF THE INVENTION

The prodrugs which are the subject of the present invention include prodrugs that contain a pharmaceutical species (X) for the treatment of a disease state and a promoiety (Y) that is covalently bound to the pharmaceutical species where the promoiety Y is an enzymatic substrate as well as a substrate for a membrane transporter. The present invention has utility as a therapeutic agent for the treatment of a variety of disease states.

An inventive prodrug enhances the bioavailability of the pharmaceutical species while specifically targeting the enzyme responsible for promoiety removal and thus pharmaceutical species release. Bioavailability is defined herein as the amount of drug systemically available in comparison to the total amount of drug delivered to an individual. Bioavailability is typically expressed as % bioavailability and is generally measured by comparing plasma levels of drug after oral administration to plasma levels of drug after intravenous administration. This definition includes first pass metabolism, that is gut and liver metabolism, which when it occurs, occurs before the drug is available systemically. Thus, highly metabolized drugs may be completely absorbed but have a bioavailability less than 100 percent. Bioavailability is directly related to the fraction of a drug absorbed or “fraction absorbed”, which refers to the percent of a total orally delivered drug dose transported or diffused across the luminal membranes of the gastrointestinal tract into the portal vein.

A prodrug according to the present invention has the general form X—Y. X includes a wide variety of pharmaceutical compounds that have accessible reactive groups to which a promoiety is covalently bonded. In a preferred embodiment, an inventive prodrug includes a pharmaceutical species X having bioavailability of 30 percent or less. Covalent bonding of a promoiety to the pharmaceutical species X enhances bioavailability of the pharmaceutical species by greater than 2 fold. An exemplary list of pharmaceutical species X that currently have clinical indications and bioavailability of 30 percent or less is described herein.

In one embodiment, the pharmaceutical species X has a bioavailability of 30 percent or less and a molecular weight ranging from 100-1000 Daltons. In another embodiment of an inventive prodrug, the pharmaceutical species X has a molecular weight ranging from 260-800 Daltons. In general, bioavailability of pharmaceutical species decreases with increasing molecular weight. Thus, it is surprising that modification of a pharmaceutical species X having higher molecular weights, such as those in the range of 260-1000 Daltons, enhances bioavailability.

In another embodiment, the pharmaceutical species is a cyclic nucleoside analog having a bioavailability of 30 percent or less.

In a further embodiment, the pharmaceutical species X includes a halogen.

Examples of pharmaceutical species (X) according to the present invention illustratively include anti-neoplastic compounds such as floxuridine, gemcitabine, cladribine, dacarbazine, melphalan, mercaptopurine, thioguanine, cis-platin, and cytarabine; and anti-viral compounds such as fludarabine, cidofovir, tenofovir, and pentostatin. Further examples of pharmaceutical species according to the invention include adenocard, adriamycin, allopurinol, alprostadil, amifostine, aminohippurate, argatroban, benztropine, bortezomib, busulfan, cacitriol, carboplatin, daunorubicin, dexamethasone, topotecan, docetaxel, dolasetron, doxorubicin, epirubicin, estradiol, famotidine, foscarnet, flumazenil, fosphenytoin, fulvestrant, hemin, ibutilide fumarate, irinotecan, levocarnitine, idamycin, sumatriptan, granisetron, metaraminol, metaraminol, methohexital, mitoxantrone, morphine, nalbuphine hydrochloride, nesacaine, oxaliplatin, palonosetron, pamidronate, pemetrexed, phytonadione, ranitidine, testosterone, tirofiban, toradol, triostat, valproate, vinorelbine tartrate, visudyne, zemplar, zemuron, and zinecard.

The promoiety Y is selected to be covalently bindable to the pharmaceutical species X, as well as simultaneously being a substrate for enzymatic cleavage and itself or as X—Y being a substrate for a membrane transporter. The promoiety Y includes synthetic and naturally occurring amino acids, di- and polypeptides, pentose sugars, hexose sugars, disaccharides, polysaccharides, C₂-C₂₀ linear or branched alkyl groups, and C₃-C₂₀ alkyl groups having a substituent where the substituent is selected from the group consisting of: amino, hydroxyl, phospho-, phosphatidyl-, and the aforementioned groups.

For the enhanced transport, there are a wide variety of known intestinal and cellular transporters that have been identified that could serve as targets for an inventive prodrug. A list of exemplary transporters is given in Table 1. Also in Table 1 is compiled a list of compounds that are known to interact with specific transporters.

TABLE 1
Transporter Targets.

| Transporters | Active species/substrates |
|---|---|
| Amino acid transporters | gabapentin, D-cyclosporin, isobutyl |
| | gaba, L-methyldopa, L-dopa, baclofen |
| Peptide transporter | β-lactam antibiotics, ACE |
| (HPEPT1, HPT1) | inhibitors, valacyclovir, |
| | valganciclovir, cyclosporin, |
| | L-methyldopa, cephalexin |
| Nucleoside transporters | zidovudine, zalcitabine cladribine |
| (CNT1 CNT2, ENT1 ENT2) | ara-C, ara-A, fludarabine, dilazep, |
| | dipyridamole, draflazine hypoxanthine |
| Organic cation transporters | tetraethylammonium, N- |
| (OCT1, ORCTL3) | methylnicotineamide, thiamine, |
| | tyramine, tryptamine, choline, |
| | spermine, spermidine, d-tubocurarine, |
| | procanamide, dobamine, noradrenaline, |
| | serotonin, istamine, corticosterone, |
| | MPP, despramine, qunidine, |
| | verapamil, midazolam |
| Organic anion transporters | methotrexate, cefodizime, ceftriaxone, |
| (MOAT (MRP2), MCT1) | pravastatin, temocaprilat, salicylic |
| | acid, p-amnobenzoic acid, benzoic |
| | acid, nicotinic acid, lactate |
| Glucose transporters | p-nitrophenyl-β-D-glucopyranoside, |
| (GLUT2, GLUT5, SGLT1, | β-D-galactopyranoside |
| SAAT1) | |
| Bile acid transporters | Thyroxine, chlorambucil, crilvastatin |
| (IBAT/ISBT) | |
| Phosphate transporters | fosfomycin, foscarnet, digoxin, |
| (NPT4, NAPI-3B, P-gp) | cyclosporin |
| Vitamin transporters | reduced vitamin C, methotrexate, |
| (SVCT1-2, folate | nicotinic acid, thiamine, vitamin |
| transporters, SMVT) | B-12, R.I.-K(biotin)-Tat9 |

The chemical synthesis of an inventive prodrug is appreciated to be largely dictated by the reactive sites available on the active species X or those incorporated therewith, and the corresponding reactive site found on the promoiety Y. By way of example, a pharmaceutical species X including or chemically modified to include a carboxylic acid group readily forms a covalent bond with a promoiety Y through conventional organic chemistry reactions. For instance, reaction of a pharmaceutical species carboxylic acid group with vinyl chloride creates an active species carbonyl chloride which upon reaction with a promoiety hydroxide or primary amine yields X—Y in the form of an ester (XCOOY) and an amide (XCONHY), respectively. In a similar fashion a pharmaceutical species containing a hydroxyl group is readily esterified through a similar reaction scheme. Additionally, an amine group found in a pharmaceutical species is readily alkylated by reaction with a promoiety halide to yield XNHY where the halide acid represents the other metathesis reaction product. Illustrative linkages between and X and Y include an ester, an amide, an ether, a secondary amine, a tertiary amine, and an oxime. While the synthesis of an inventive prodrug is detailed above with chemistry being performed on the pharmaceutical species X in order to form a covalent bond with a subsequent reactant promoiety Y, it is appreciated that modifying chemistry is readily performed on the promoiety Y followed by subsequent reaction with active species X. Additionally, it is appreciated that protecting agents are operative herewith to preclude reaction at one or more active sites within a pharmaceutical species X and/or promoiety Y during the course of a coupling reaction. Additionally, a deprotecting agent is operative herein to convert a pharmaceutical species X and/or a promoiety Y into a reactive thiol, amine or hydroxyl substituent. Protecting agents and deprotecting agents are well known in the art. Theodore W. Green and Peter G. M. Wets, Protective Groups in Organic Synthesis, 2^{nd} Edition (1991).

In a preferred embodiment, an inventive composition includes a prodrug having the general formula X—Y wherein an active species X is a pharmaceutical species characterized by lack of bioavailability when administered orally to an individual. In this embodiment, promoiety Y is an amino acid having a covalent bond to the pharmaceutical species. In a preferred embodiment, an inventive prodrug includes a pharmaceutical species having bioavailability of 30 percent or less. Covalent bonding of a promoiety to the pharmaceutical species enhances bioavailability of the pharmaceutical species by greater than 2 fold.

Naturally-occurring or non-naturally occurring amino acids are used to prepare the prodrugs of the invention. In particular, standard amino acids suitable as a prodrug moiety include valine, leucine, isoleucine, methionine, phenylalanine, asparagine, glutamic acid, glutamine, histidine, lysine, arginine, aspartic acid, glycine, alanine, serine, threonine, tyrosine, tryptophan, cysteine and proline. Particularly preferred are L-amino acids. Optionally an included amino acid is an alpha-, beta-, or gamma-amino acid. Also, naturally-occurring, non-standard amino acids can be utilized in the compositions and methods of the invention. For example, in addition to the standard naturally occurring amino acids commonly found in proteins, naturally occurring amino acids also illustratively include 4-hydroxyproline, γ-carboxyglutamic acid, selenocysteine, desmosine, 6-N-methyllysine, ε-N,N,N-trimethyllysine, 3-methylhistidine, O-phosphoserine, 5-hydroxylysine, ε-N-acetyllysine, ω-N-methylarginine, N-acetylserine, γ-aminobutyric acid, citrulline, ornithine, azaserine, homocysteine, β-cyanoalanine and S-adenosylmethionine. Non-naturally occurring amino acids include phenyl glycine, meta-tyrosine, para-amino phenylalanine,3-(3-pyridyl)-L-alanine, 4-(trifluoromethyl)-D-phenylalanine, and the like.

In one embodiment of an inventive compound, the amino acid covalently coupled to the pharmaceutical species is a non-polar amino acid such as valine, phenylalanine, leucine, isoleucine, glycine, alanine and methionine.

In a further embodiment, more than one amino acid is covalently coupled to the pharmaceutical species. Preferably, a first and second amino acid are each covalently coupled to separate sites on the pharmaceutical species. Optionally, a dipeptide is covalently coupled to the pharmaceutical species.

An inventive prodrug is metabolized in the individual to yield the pharmaceutical species and an amino acid. For example, endogenous esterases cleave a described inventive prodrug to yield the pharmaceutical species and amino acid. Table 2 details a nonlimiting list of activation enzymes that are operative to activate various embodiments of prodrugs X—Y by removal of the prodrug moiety Y.

TABLE 2
Activation Enzymes for Inventive Prodrugs.
α/β hydrolase fold family

| | |
|---|---|
| Acylaminoacyl peptidase | Oligopeptidase B |
| (EC 3.4.19.1) | (EC 3.4.21.83) |
| Prolyl oligopeptidase | Biphenyl hydrolase-like |
| (EC 3.4.21.26) | enzyme |
| lecithin: cholesterol | epoxide hydrolase |
| acyltransferase | |
| dipeptidyl peptidase IV (DPP TV) | |

Other peptidases

| | |
|---|---|
| prolidase | prolyl aminopeptidase |
| metalloendopeptidases | tripeptidyl peptidase II |

Alkaline phosphatases and other esterases

| | |
|---|---|
| carboxylesterase | carboxylesterase |
| palmitoyl protein thioesterase | esterase D |
| intestinal alkaline phosphatase | |

Cytochrome p450s

| | |
|---|---|
| cytochrome P450IIA3 (CYP2A3) | cytochrome P(I)-450 |
| cytochrome P450 (CYP2A13) | cytochrome P-450 (P-450 HFLa) |
| cytochrome P450-IIB (hIIB1) | cytochrome P450 4F2 (CYP4F2) |
| cytochrome P4502C9 (CYP2C9) | vitamin D3 25-hydroxylase |
| cytochrome P4502C18 (CYP2C18) | lanosterol 14-demethylase |
| cytochrome P4502C19 (CYP2C19) | cytochrome P450 (CYP51) |
| cytochrome P-450IID | cytochrome P450 reductase |
| cytochrome P450 monooxygenase | cytochrome P450 PCN3 gene |
| CYP2J2 | |

In some embodiments of an inventive compound, cleavage of the bond between X and Y yields an inactive pharmaceutical species which is further metabolized in vivo to achieve the active pharmaceutical species. For example, 6-mercaptopurine and 6-thioguanine are each inactive and require phosphorylation by the enzyme hypoxanthine-guanine phosphoribosyltransferase for transformation to the active cytotoxic form.

It is appreciated that prodrugs according to the present invention are readily created to treat a variety of diseases illustratively including metabolic disorders, cancers, and gastrointestinal disease. In a preferred embodiment, an inventive prodrug is formulated for administration to a human individual, and bioavailability and fraction absorbed measurements refer to measurements made in humans. However, it is appreciated that an inventive prodrug and method of treatment may be indicated in non-human applications as well. Thus, an inventive prodrug is advantageously administered to a non-human organism such as a rodent, bovine, equine, avian, canine, feline or other such species wherein the organism possesses an enzyme and a membrane transporter for which the prodrug is a substrate.

A method of treatment according to the present invention includes administering a therapeutically effective amount of an inventive prodrug to an organism possessing an enzyme and a membrane transporter wherein the prodrug is a substrate for both.

In a particular embodiment of an inventive method for delivering a pharmaceutical species to an individual the method includes the step of administering an inventive prodrug as described herein to the gastrointestinal lumen of an individual. Particularly preferred is a prodrug which includes a pharmaceutical species characterized by bioavailability of 30 percent or less, a molecular weight in the range of 100-1000 Daltons, and wherein the pharmaceutical species is not acyclovir, ganciclovir, BRL44385, or penciclovir. An amino acid is included which has a covalent bond to the pharmaceutical species. The prodrug is transported from the gastrointestinal lumen by a specific transporter and enzymatically cleaved to yield the pharmaceutical species, thereby delivering the pharmaceutical species to the individual.

Variable dosing regimens are operative in the method of treatment. While single dose treatment is effective in producing therapeutic effects, it is noted that longer courses of treatment such as several days to weeks have previously been shown to be efficacious in prodrug therapy (Beck et al., Human Gene Therapy, 6:1525-30 (1995)). While dosimetry for a given inventive prodrug will vary, dosimetry will depend on factors illustratively including target cell mass, effective active species X cellular concentration, transporter efficiency, systemic prodrug degradation linetics, and secondary enzymatic cleavage that reduces active species lifetime. It is appreciated that conventional systemic dosimetry is not applicable to the present invention.

A prodrug is administered by a route determined to be appropriate for a particular subject by one skilled in the art. For example, the prodrug is administered orally; parenterally, such as intravenously; by intramuscular injection; by intraperitoneal injection; intratumorally; transdermally; or rectally. The exact dose of prodrug required is appreciated to vary from subject to subject, depending on the age, weight and general condition of the subject, the severity of the disease being treated, the particular pharmaceutical species, the mode of administration, and the like. An appropriate dose is readily determined by one of ordinary skill in the art using only routine experimentation given the teachings herein. Generally, dosage is in the range of about 0.5-500 mg per m².

Depending on the intended mode of administration, the prodrug can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. Time-release preparations are specifically contemplated as effective dosage formulations. The compositions will include an effective amount of the selected substrate in combination with a pharmaceutically acceptable carrier and, in addition, may include other medicinal agents, pharmaceutical agents, carriers, or diluents. Further, a prodrug may be formulated as a pharmaceutically acceptable salt.

For solid compositions, conventional nontoxic solid carriers include, for example, pharmaceutical grades of mannitol, lactose, starch, magnesium stearate, sodium saccharine, talc, cellulose, glucose, sucrose and magnesium carbonate. Liquid pharmaceutically administrable compositions can, for example, be prepared by dissolving or dispersing an active compound with optimal pharmaceutical adjuvants in an excipient, such as water, saline, aqueous dextrose, glycerol, or ethanol, to thereby form a solution or suspension. If desired, the pharmaceutical composition to be administered may also contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art; for example, see Remington's The Science and Practice of Pharmacy (20^{th} Edition).

For oral administration, fine powders or granules may contain diluting, dispersing, and/or surface active agents, and may be presented in water or in a syrup, in capsules or sachets in the dry state or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration is generally by injection. Injectables can be prepared in conventional forms, either liquid solutions or suspensions, solid forms suitable for solution or prior to injection, or as suspension in liquid prior to injection or as emulsions.

Ara A, also referred to as adenine 9-beta-D-arabinofuranoside and by the trade name vidarabine is an antiviral and anti-cancer compound characterized by poor bioavailability and particularly by poor bioavailability when administered orally.

For example, Ara A is characterized by low lipophilicity and thus shows low intestinal membrane permeability. Ara A is readily metabolized by adenosine deaminase (ADA) to ara-hypoxanthine (ara-H) which has very low antiviral activity. Adenosine deaminase is expressed at significant levels in intestinal mucosa, for example as described in Singhal, D. et al., J. Pharm. Sci. 87(5):578-585; and Singhal, D. et al., J. Pharm. Sci. 87(5):569-577, a factor contributing to p bioavailability of Ara A.

Ara A is poorly soluble in aqueous solutions, such that formulation options for parenteral and peroral administration are limited.

Prodrug Ara A compositions are provided according to embodiments of the present invention having greater bioavailability than unmodified Ara A. In particular, embodiments of prodrug Ara A compositions are characterized by increased transport by an intestinal transporter compared to Ara A; increased stability in the presence of adenosine deaminase compared to Ara A; and/or increased solubility in aqueous solution compared to Ara A.

A composition is provided according to embodiments of the present invention including an Ara A prodrug having the structural formula: 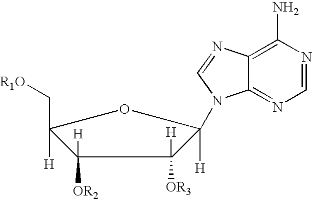
where R₁, R₂ and R₃ are each independently H, or a substrate for a transporter selected from: an amino acid, a dipeptide and a tripeptide, where at least one of R₁, R₂ and R₃ is an amino acid, a dipeptide or a tripeptide. An included amino acid is an L-amino acid and/or a D-amino acid in particular embodiments of an inventive prodrug.

Optionally, R₁ is an amino acid, a dipeptide or a tripeptide, and R₂ and R₃ are each independently H, an amino acid, a dipeptide or a tripeptide.

In a further option, R₁ is an amino acid, a dipeptide or a tripeptide, and R₂ and R₃ are both H.

Particular examples of Ara A prodrugs according to the present invention include Ara A linked to a non-polar amino acid through an ester linkage.

Specific Ara A prodrugs of the present invention include 5′-O-D-isoleucyl Ara A; 5 ′-O-L-isoleucyl Ara A; 5′-O-D-valyl-O Ara A; 5′-O-L-valyl-O Ara A; 5′-O-glycyl-O Ara A; 5′-O-D-phenylalanyl Ara A; 5′-O-L-phenylalanyl Ara A; 5′-O-D-leucyl Ara A; 5′-O-L-leucyl Ara A; 5′-O-L-aspartyl Ara A; 5′-O-D-alpha-aspartyl Ara A; 5′-O-L-alpha-aspartyl Ara A; 5′-O-D-beta-aspartyl Ara A; 5′-O-L-beta-aspartyl Ara A; and 5′-O-L-prolyl Ara A.

An example of a dipeptide Ara A prodrug is 5′-O-L-alagly Ara A.

While both D-amino acid and L-amino acid conjugates of Ara A are prodrugs which demonstrate improved bioavailability compared to unmodified Ara A, conjugation of a D-amino acid to Ara A results in prodrugs according to particular embodiments of the present invention having unexpectedly improved bioavailability compared to prodrugs including L-amnino acids. D-amino acids generally have lower affinity than L-amino acids for the intestinal dipeptide transporter such that reduced transport of the D-amino acid Ara A prodrug and reduced bioavailability compared to the L-amino acid Ara A prodrug might be expected. For example, as shown in Examples herein, administration of 5′-O-D-valyl-Ara A results in concentrations of the prodrug in systemic plasma which are higher over a longer period of time than concentrations of 5′-O-L-valyl-Ara A following administration of similar amounts of these prodrugs. Thus, in particular embodiments, an Ara A prodrug includes a D-amino acid.

In preferred embodiments, the amino acid, dipeptide or tripeptide substrate for a transporter is a substrate for an intestinal transporter. In particular embodiments, an amino acid included in an inventive prodrug includes an amino acid which is a substrate for a nutrient transporter such as PEPT1 and/or HPT1.

In a preferred embodiment of an inventive composition, a prodrug is characterized by at least two-fold greater bioavailability compared to adenine 9-beta-D-arabinofuranoside, Ara A.

Also provided is a composition including a prodrug having the structural formula: 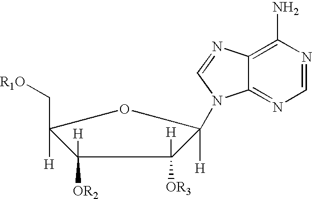
where R₁ is an amino acid and where R₂ and R₃ are both H.

Examples of specific prodrugs include 5′-O-D-isoleucyl Ara A; 5′-O-L-isoleucyl Ara A; 5′-O-D-valyl Ara A; 5′-O-L-valyl Ara A; 5′-O-glycyl Ara A; 5′-O-D-phenylalanyl Ara A; 5′-O-L-phenylalanyl Ara A; 5′-O-D-leucyl Ara A; 5′-O-L-leucyl Ara A; 5′-O-L-aspartyl Ara A; 5′-O-D-alpha-aspartyl Ara A; 5′-O-L-alpha-aspartyl Ara A; 5′-O-D-beta-aspartyl Ara A; 5′-O-L-beta-aspartyl Ara A; and 5′-O-L-prolyl Ara A.

Ara A prodrugs according to the present invention are readily created to treat a variety of diseases. In particular embodiments, an Ara A prodrug is administered to a subject in need thereof as an antiviral agent, for example, to treat a viral infection. In further particular embodiments, an Ara A prodrug is administered to a subject in need thereof as an antiproliferative agent, for example, to treat cancer such as a tumor or other pathological cell proliferative disorder.

A method of treatment according to the present invention includes administering a therapeutically effective amount of an inventive Ara A prodrug to an organism possessing an enzyme and a membrane transporter wherein the Ara A prodrug is a substrate for both.

In a particular embodiment of an inventive method for delivering Ara A to an individual the method includes the step of administering an inventive Ara A prodrug as described herein to the gastrointestinal lumen of an individual subject. The Ara A prodrug is transported from the gastrointestinal lumen by a specific transporter and enzymatically cleaved to yield Ara A, thereby delivering Ara A to the individual subject.

An antiviral and/or antiproliferative method is provided according to embodiments of the present invention which includes administering to a subject in need thereof a therapeutically effective amount of a composition comprising an Ara A prodrug having the structural formula: 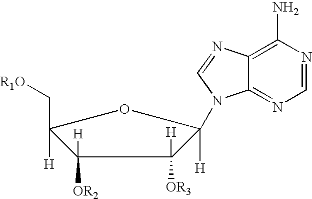
where R₁, R₂ and R₃ are each independently H, an amino acid, a dipeptide or a tripeptide, where at least one of R₁, R₂ and R₃ is an amino acid, a dipeptide or a tripeptide; and a pharmaceutically acceptable carrier,

In particular embodiments, an antiviral method includes administration of an inventive composition to a subject infected or at risk of infection with a virus from a virus family such as Adenoviridae, Arenaviridae, Bunyaviridae, Caliciviridae, Coronaviridae, Flaviviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, and Togavilidae.

In a further particular embodiment, an antiviral method includes administration of an inventive composition to a subject infected or at risk of infection with a virus from the virus family Poxviridae, including small pox virus, cow pox and vaccinia virus.

A preferred antiviral and/or antiproliferative method includes oral administration of an inventive Ara A prodrug composition to a subject.

In particular embodiments, a method of treating a subject includes administration of a prodrug compound selected from 5′-O-D-isoleucyl Ara A; 5′-O-L-isoleucyl Ara A; 5′-O-D-valyl-Ara A; 5′-O-L-valyl Ara A; 5′-O-glycyl Ara A; 5′-O-D-phenylalanyl Ara A; 5′-O-L-phenylalanyl Ara A; 5′-O-D-leucyl Ara A; 5′-O-L-leucyl Ara A; 5′-O-L-aspartyl Ara A; 5′-O-D-alpha-aspartyl Ara A; 5′-O-L-alpha-aspartyl Ara A; 5′-O-D-beta-aspartyl Ara A; 5′-O-L-beta-aspartyl Ara A; 5′-O-L-prolyl Ara A; and a combination of any of these.

Methods of synthesizing an Ara A prodrug are provided herein. Broadly described, embodiments of a method of synthesizing an Ara A prodrug include protecting one or more reactive groups of Ara A, conjugating a promoiety to Ara A and deprotecting the protected reactive group or groups, resulting in a promoiety-Ara A conjugate prodrug.

Exemplary methods of synthesizing an Ara A prodrug are described in detail in Examples herein.

An Ara A prodrug according to the present invention is provided as the free base in particular embodiments. Optionally, an Ara A prodrug according to the present invention is provided as a pharmaceutically acceptable salt. For example, an inventive Ara A prodrug is illustratively provided as a salt of an inorganic or organic acid such as hydrochloric acid, sulfuric acid, phosphoric acid, acetic acid, citric acid, benzoic acid, maleic acid, funaric acid, succinic acid, methanesulfonic acid, p-toluenesulfonic acid or trifluoroacetic acid. Further optionally, an Ara A prodrug is provided as a hydrate.

The examples presented below are intended to illustrate a particular embodiment of the invention and is not intended to limit the scope of the specification, including the claims, in any way.

### EXAMPLES

### Example 1

Method for Synthesis of Floxuridine Prodrugs

Floxuridine is fluorinated pyrimidine compound that is currently used as an anti-neoplastic anti-metabolite. The drug is absorbed orally to a certain extent, but the absolute bioavailability shows high variability (Van Der Heyden S A, Highley M S, De Bruijn E A, Tjaden U R, Reeuwijk H J, Van Slooten H, Van Oosterom A T, Maes R A. Pharmacokinetics and bioavailability of oral 5′-deoxy-5-fluorouridine in cancer patients Br J Clin Pharmacol. 1999 Apr; 47(4):351-6.). To address the question of targeting of drugs to specific transporters within the intestine and targeted activation, a number of floxuridine amino acid ester prodrugs are synthesized, as shown in the figure.

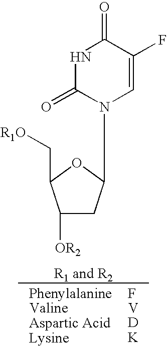

The 3′-monoester, 5′-monoester, and 3′,5′-diesterprodrugs of floxuridine are synthesized as follows: N-t-Boc-amino acid (1.8 mmole), dimethyl-pyrindin-4-yl-amine (0.19 mmole) and dicyclohexyl carbodiimide (2.17 mmole) are added to floxuridine (1.33 mmole) in dry dimethylformamide (DMF) (30 ml). The solution is stirred under a nitrogen atmosphere at ambient temperature for 48 hrs and then the mixture is filtered. The DMF is removed from the filtrate in vacuo and the residue is chromatographed on silica gel, using CH₃OH/CH₂Cl₂ (1:4) as the eluant. After evaporation of the desired fractions, the resulting white solid intermediate is dissolved in 10 ml of freshly distilled trifluoroacetic acid/CH₂Cl₂ (1:1) and stirred at 0° C. for 30 min. The excess acid is removed in vacuo. The residue is freeze-dried to obtain the desired prodrug as a hygroscopic, fluffy white solid. The structures are confirmed by ¹H-NMR, ¹³C-NR and LC/MS/MS spectrometer. For each amino acid of Equation 1, three prodrugs were synthesized: a 5′ ester, a 3′ ester and a 5′,3′ ester. The structures are confirmed by ¹H-NMR, ¹³C-NMR and LC/MS/MS spectrometer.

### Example 2

Method of Synthesis for Melphalan Prodrugs

Melphalan is a phenylalanine derivative of nitrogen mustard, a bifunctional alkylating agent active against certain human neoplastic diseases. It is absorbed orally to a certain extent, but the oral bioavailability shows high variability (Physicians Desk Reference 57^{th} edition, Thompson PDR, Montvale, N.J.). A prodrug of the melphalan containing an additional amino acid can be synthesized to increase the bioavailability of the melphan and to aid in the targeting of the melphalan to the tumor tissue. An amino acid prodrug of the melphalan using proline as the amino acid is using a 4 step process.

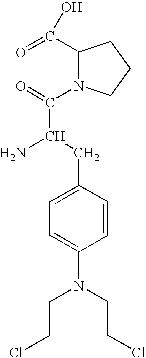

First, t-Boc protected L-melphalan, 2 is synthesized by adding di-tert-butyl dicarbonate (196 mg, 0.89 mmol) to an ice-cold solution of melphalan (1-250 mg, 0.82 mmol) in a mixture of dioxane (2 mL), distilled water (1 mL), and 1N NaOH (1 mL). The mixture is stirred for 1 hour at 0° C. and then for 16 hour at room temperature. After the reaction is complete, the mixture is concentrated and ethyl acetate and distilled water are added. The pH of the mixture is adjusted to 2 with hydrochloric acid and the aqueous phase is then extracted with ethyl acetate (3 times with 15 ml). The combined organic phases are washed with distilled water and brine, dried over MgSO4, and the filtrate is concentrated under vacuum to yield compound 2 (330 mg, yield 98%).

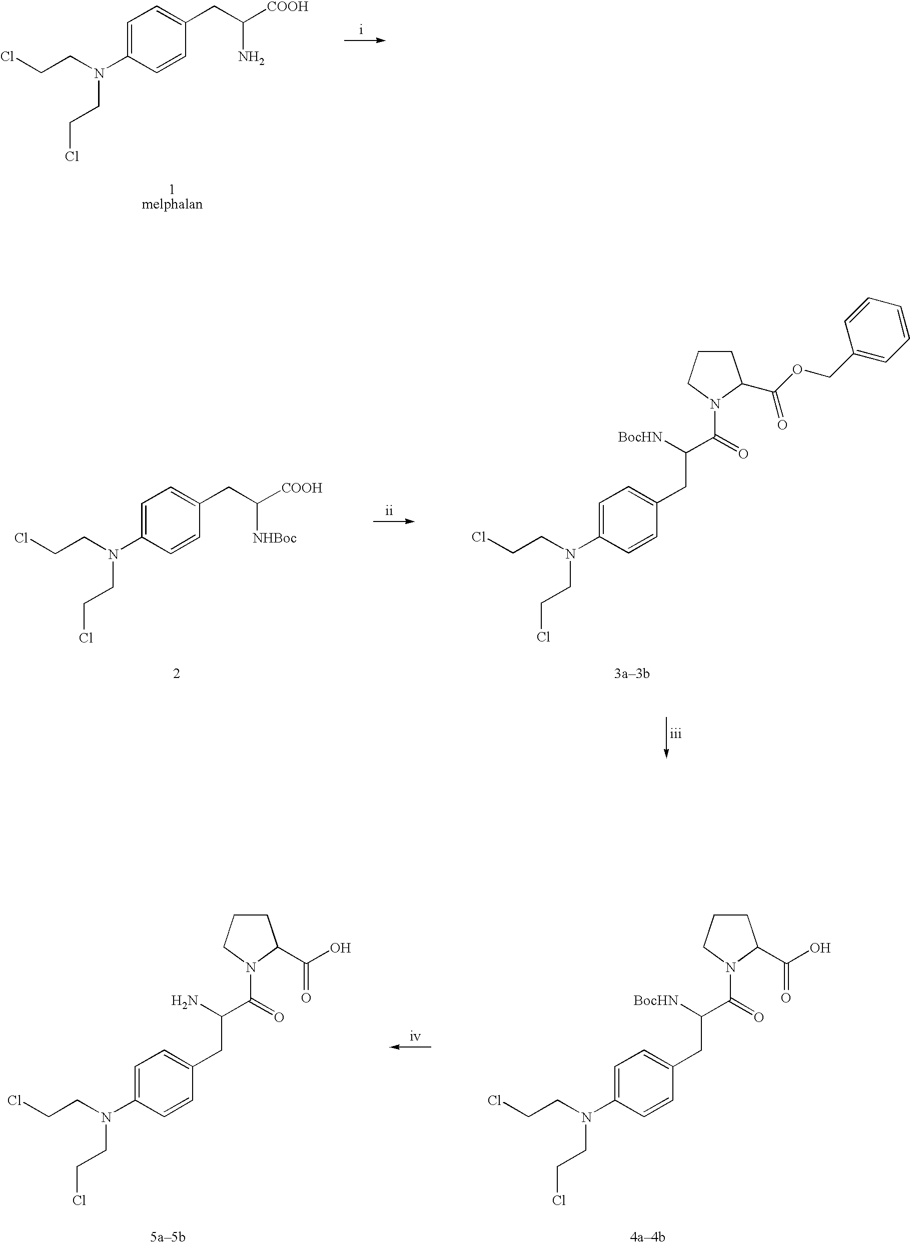

In the second step, 4-[bis(2-chloroethyl) butyloxycarbonyl]-L-phenylalanyl-L-proline benzyl ester, 3a, is synthesized by addition of compound 2 (330 mg, 0.82 mmol) to 0° C. solution of L-proline benzyl ester hydrochloride (197 mg, 0.82 mmol) dissolved in chloroform (15 mL) and triethylamine (0.14 mL). Dicyclohexylcarbodiimide (DCC, 165 mg, 0.82 mmol) is added to the mixture and it is stirred for 3 hour at 0° C., allowed to warm to room temperature and stirred for an additional 24 h. The reaction mixture is filtered and the chloroform removed under reduced pressure. The residue is extracted with ethyl acetate and washed with distilled water and brine. The organic layer is dried over MgSO4 and concentrated under vacuum. The residue is subjected to column chromatography to yield compound 3a (545 mg, yield 75%). In the third step, compound 3a (520 mg, 0.88 mmol) is dissolved in 15 ml of anhydrous ethanol and 80 mg of 10% Pd/C is added. The mixture is vigorously stirred under hydrogen at room temperature for 12 h. The catalyst is removed by filtration through a bed of celite and washed with ethanol. The resulting filtrate is concentrated under vacuum and 4-[bis(2-chloroethyl)butyloxycarbonyl]-L-phenylalanyl-L-proline, 4a, is purified using a silica gel column eluted using a graded series of methylene chloride/methanol mixtures (ratios graded from 10:1 to 1:1) as the elutant. In the final step, a solution of 4a (300 mg, 0.6 mmol) in 5 ml hydrogen chloride-saturated dioxane is stirred for 25 min at 20° C. The mixture is concentrated under vacuum and the residue washed with pentane, to yield 4-[bis(2-chloroethyl)]-L-phenylalanyl-L-proline, 5a as the hydrochloride salt (210 mg, yield 80%). In this example, 4-[bis(2-chloroethyl)]-L-phenylalanyl-D-proline, 5b is synthesized by substituting D-proline benzyl ester (3b) in place of 3a and following the steps outlined above. The structures are confirmed by ¹H-NMR, ¹³C-NMR and LC/MS/MS spectrometer.

### Example 3

Synthesis of the Poorly Absorbed Nucleoside Prodrugs: Cladribine and Gemcitabine

Gemcitabine is a pyrimidine nucleoside analog and cladribine is a purine nucleoside analog. These drugs are both useful as anticancer agents. However, both drugs show very low oral bioavailability and are administered by i.v. infusion. To aid the oral pharmacokinetic and pharmacodynamic profile of the drugs such that they could be used in an oral drug product, amino acid prodrugs of these nucleoside analog drug that target the intestinal transporters can be synthesized using a two-step process. An example of the synthetic route is shown to make valyl, isoleucyl, and phenylalanyl prodrugs of Gemcitabine. Similar reaction amounts and steps can be used to synthesize the cladribine prodrugs.

In the first step, Boc protected amino acids (Boc-L-Val-OH, Boc-D-Val-OH, Boc-L-Phe-OH, Boc-D-Phe-OH, or Boc-L-Ile-OH) (1.5 mmol), dicyclohexylcarbodiimide (DCC) (1.5 mmol) and dimethylaminopyridine (DMAP) (0.15 mmol) are reacted with gemcitabine (1.5 mmol) in 10 ml of dry N,N-dimethylformamide (DMF). The reaction mixture is stirred at room temperature for 24 h. Each reaction yields three products (3′ and 5′ monoesters and 3′,5′ diesters). The reaction mixture is filtered and the DMF is removed in vacuo at 50-55° C. The residue is dissolved in ethyl acetate (30 ml) and is washed with water (2×20 ml), saturated NaHCO₃ (2×20 ml), and brine (1×20 ml). The organic layer is dried over MgSO₄ and concentrated in vacuo. The three intermediates are purified using silica gel column chromatography, which is eluted with a graded series of ethyl acetate: hexane mixtures (ethyl acetate: hexane, 1:1-1:0) as the elutant.

In the second step, the blocking group is removed from the purified intermediates by treating with 4 ml of TFA:DCM:water (6:3:1) for 4 hours. Finally, the solvent is removed under vacuum and the residue is reconstituted in water and freeze dried. The combined yield of gemcitabine prodrugs is approximately 40%. The structures are confirmed by ¹H-NMR and LC/MS/MS spectrometer.

### Example 4

Method of Synthesis for Cidofovir Prodrugs

Cidofovir is a polar antiviral agent that exhibits very poor oral bioavailability. In order to increase the oral bioavailability of cidofovir, amino acid ester prodrugs are synthesized. In one embodiment, these prodrugs are synthesized through a modification of synthetic schemes for the synthesis of the parent drug, cidofovir. (Brodfuehrer, P. R. e.a., A Practical Synthesis of (S)-HPMPC.Tet Lett, 1994. 35(20): p. 3243-3246; and Vemishetti, P., P. R. Brodfuehrer, H. Howell, and S. C., Process for the preparation of nucleotides. 1995, Institute of Organic Chemistry and Biochemistry of the Academy of New York: USA.). As shown in scheme 1, the phosphono group remains free whereas in the second example, shown in scheme 2, the phosphono group is ethylated. In both cases amino acids are attached to the free hydroxyl group of cidofovir.

Cidofovir amino acid prodrugs with free phosphate hydroxyl groups are synthesized as described in Scheme 1. Briefly, the free amine of cytosine is protected with tert-butyloxycarbonyl (Boc) group. The Boc protected cytosine is coupled to Mtt (4-methyltrityl) protected (R)-glycidol (1), in presence of catalytic amount of sodium hydride in DMF at 105° C. for 5 hour to yield compound 6. Reaction of 6 with dibenzyltosyloxymethylphosphonate (4) in presence of NaH yields the nucleotide ester (7). Removal of the Mtt group by 50% acetic acid in DCM gives the corresponding alcohol (8). The free hydroxyl group of 8 is coupled to N-tBoc-protected amino acids in presence of N,N′-dicyclohexylcarbodiimide (DCC) and dimethylamino pyridine (DMAP). The resulting Boc protected amino acid esters of cidofovir (9) is purified by column chromatography. Boc and benzyl groups are cleaved simultaneously by treating the purified material (9) with 90% trifluoroacetic acid (TFA) for 4 h. After evaporation of TFA, the residue is reconstituted with water and lyophilized. The amino acid prodrugs of cidofovir (10) are obtained as TFA salts.

Cidofovir amino acid prodrugs that also have the phosphono hydroxyls protected by ethyl groups are synthesized as described in Scheme 2. Briefly, the free amine of cytosine is protected with benzyloxy-carbonyl (Z) group. The Z protected cytosine is coupled to Mtt (4-methyltrityl) protected (R)-giycidol (1), in presence of catalytic amount of sodium hydride in DMF at 105° C. for 5 hour to yield compound 11. Reaction of 11 with diethyl-tosyloxymethylphosphonate (5) in the presence of NaH gives the nucleotide ester (12). Removal of the Mtt group by 50% acetic acid in DCM yields the corresponding alcohol (13). The free hydroxyl group of 13 is coupled to N-Z-protected amino acids in the presence of N,N′-dicyclohexyl-carbodiimide (DCC) and dimethylamino pyridine DMAP). The resulting Z protected amino acid esters of cidofovir (14) is purified by column chromatography. The henzyl groups are cleaved by treating the purified material (14) by hydrogenation in presences of Palladium (O). After filtration and evaporation of solvents the residue is reconstituted with water and lyophilized. The amino acid prodrugs of cidofovir (15) are obtained as HCl salts.

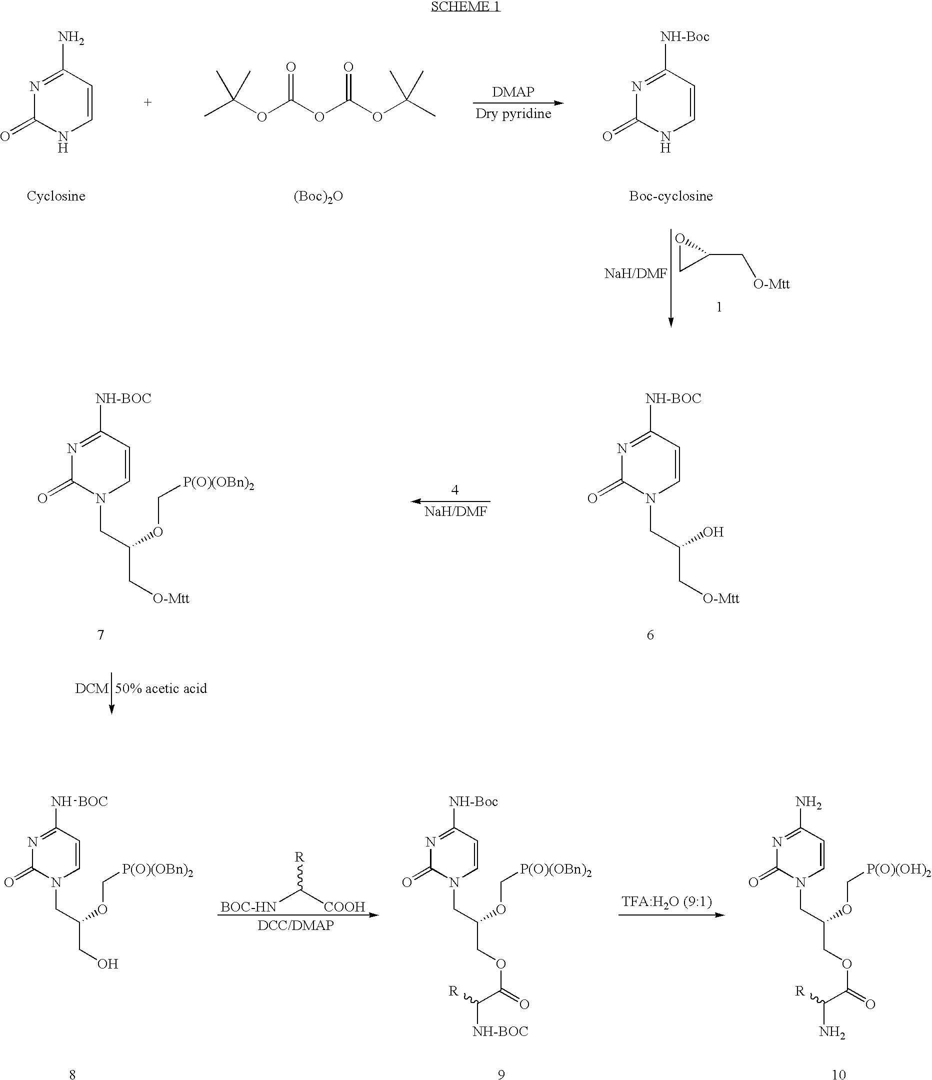

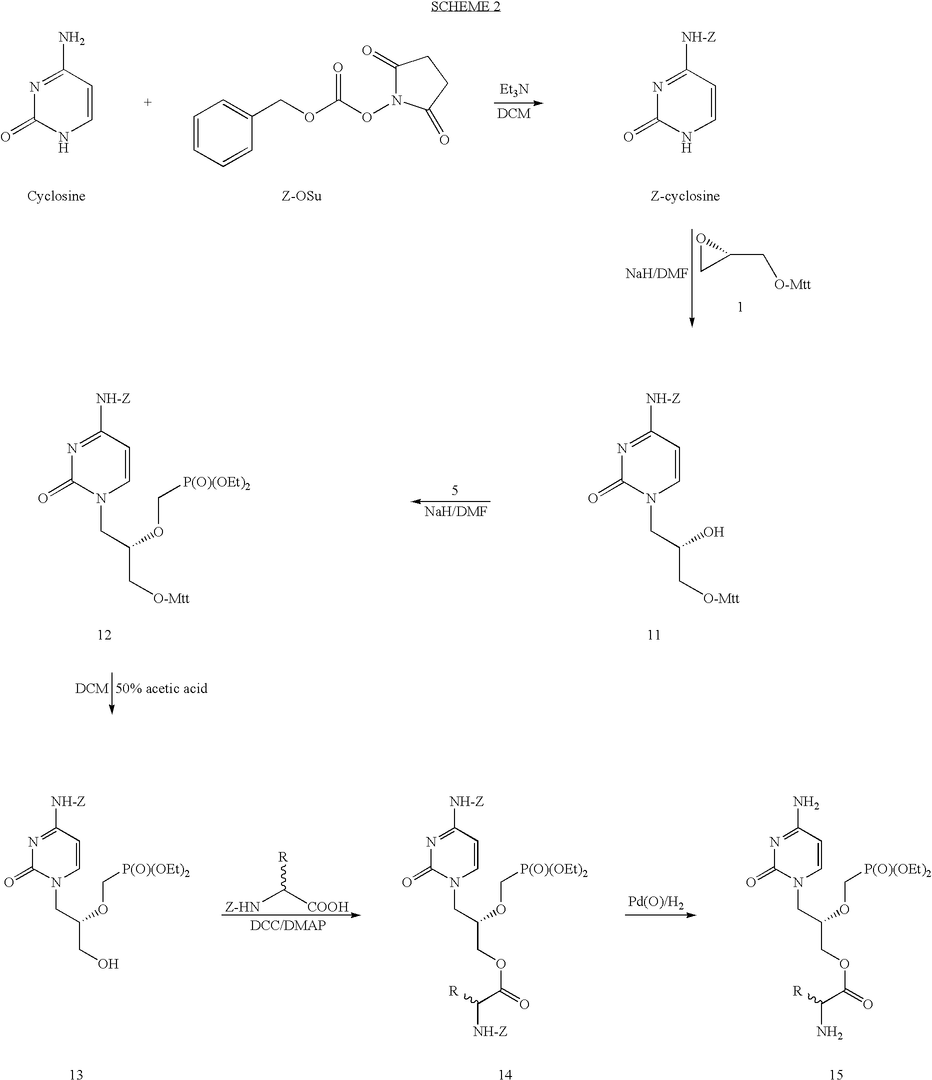

### Example 5

Determination of Binding Affinity of Amino Acid Prodrugs for the Intestinal Peptide Transporter

Amino acid prodrugs are tested for their interaction with the dipeptide transporter, HPEPT1, using tissue culture cells that are engineered to overexpress HPEPT1. In this example, the cells that overexpress HPEPT1, termed DC5, are a human meduloblastoma cell line that is stably transfected with a eukaryotic expression vector encoding HPEPT1. In this assay, the ability of the prodrug to competitively inhibit the uptake of a known substrate of HPEPT1 is measured, The known substrate is the dipeptide Glycine-Sarcosine (Gly-Sar) that has a radioactive label. DC5 cells are plated at a density of 12,000 cells/well in 96-well tissue culture plates and allowed to grow for 2 days. The cells are washed once with 200 microliters of uptake buffer and aspirated. The plates are cooled to 4° C. and 25 ul of uptake buffer containing 125 nanomoles Gly-Sar (at a specific activity of 1 microcurie/micromole) is added. The uptake buffer also contains the prodrugs to be tested at concentrations ranging from 10 micromolar to 20 millimolar. The assay is initiated by placing the plate in a shaking water bath at 37° C. and is terminated after 10 min by rapid washing with multiple changes of 4° C. phosphate buffered saline (PBS). The radioactive Gly-Sar peptide that is transported by the hpept1 is extracted from the cell layer with 200 ul of a one to one mixture of methanol and water and is counted in 4 ml of CytoScint ES™ scintillation cocktail (ICN). The data are plotted as % Gly-Sar uptake of control (no competitive substrate) versus the competitive substrate concentration. The IC50, defined as that concentration which inhibits 50% of the uptake of the Gly-Sar uptake, indicates the degree of affinity that the test prodrug has for the hpept1. Typically, values that are below 10 mM indicate that the drug interacts with transporter. The results from this experiment using a variety of prodrug compounds is given in Table 3.

TABLE 3
Affinity of prodrugs of acyclovir, ganciclovir, floxuridine,
gemcitabine and melphalan for HPEPT1 in cells that overexpress
the HPEPT1 intestinal transporter

| Compound | DC5 IC₅₀ mM |
|---|---|
| Val-acyclovir | 0.423 |
| Acyclovir | >25 |
| Val-ganciclovir | 5.23 |
| Ganciclovir | >20 |
| 3′,5′-di-O-phenylalanyl-Floxuridine | 2.78 |
| 3′-O-phenylalanyl-Floxuridine | 3.48 |
| 5′-O-phenylalanyl-Floxuridine | 3.7 |
| 3′,5′-di-O-valyl-Floxuridine | 1.6 |
| 3′-O-valyl-Floxuridine | 0.98 |
| 5′-O-valyl-Floxuridine | 1.17 |
| 3′,5′-di-O-prolyl-Floxuridine | >>20 |
| 3′-O-prolyl-Floxuridine | >>20 |
| 5′-O-prolyl-Floxuridine | >>20 |
| 3′,5′-di-O-aspartyl-Floxuridine | 9.55 |
| 3′-O-aspartyl-Floxuridine | 10.5 |
| 5′-O-aspartyl-Floxuridine | 8.30 |
| Floxuridine | >>20 |
| 3′,5′ Val-O-Gemcitabine | 1.72 |
| 3′ Val-O-Gemcitabine | 3.69 |
| 5′ Val-O-Gemcitabine | 0.70 |
| 3′,5′ Ile-O-Gemcitabine | 0.82 |
| 3′ Ile-O-Gemcitabine | 2.18 |
| 5′ Ile-O-Gemcitabine | 0.61 |
| Gemcitabine | >>20 |
| Mel-Pro | 0.17 |

The data show that addition of HPEPT1 targeting promoieties to a variety of drugs can improve the affinity of the drug for the HPEPT1 intestinal transporter.

### Example 6

Determination of Prodrug Uptake Mediated by Intestinal Transporter

Hela cells that overexpress hpept1 are incubated with a series of floxuridine prodrugs at a concentration of 50 micromolar in pH 6.0 uptake buffer for 45 minutes. The uptake reaction is stopped by washing of the cells with ice cold PBS three times. The cell layers are collected, the cells lysed, and the amount of parent and prodrug in the cell lysate are determined by high performance liquid chromatography (HPLC). The uptake experiments are repeated in control cultures that do not overexpress the hpept1. The ratio of the test versus control values provides a measure of uptake efficiency for the prodrug by the hpept1 transporter. As seen in Table 4, the 5′ floxuridine and gemcitabine prodrugs show the greatest enhancement of hpept1-mediated uptake. For the floxuridine prodrugs, the phenyl and valyl diester prodrugs show moderate uptake enhancement and the 3′ monoester prodrugs show poor uptake enhancement. For the gemeitabine prodrugs, the 5′ esters of valine and isoleucine showed the greatest enhancement of uptake. For these drugs, the 3′ and 3′,5′ diesters prodrugs showed little or no enhancement of uptake. Stereochemistry was also very important with regard to uptake. Thus, d-amino acids showed virtually no enhancement of uptake.

TABLE 4
Uptake of floxuridine and Gemcitabine Prodrugs in HeLa cells that overexpress HPEPT1.

| | Uptake (hPepT1) | Uptake Control | |
|---|---|---|---|
| A. Anticancer Prodrugs | nmole/mg/45 min | nmole/mg/45 min | hPepT1/Control |
| 3′,5′-di-O-phenylalanyl-Floxuridine | 2.96 ± 0.13* | 0.31 ± 0.02 | 9.53 |
| 3′-O-phenylalanyl-Floxuridine | 0.83 ± 0.19* | 0.32 ± 0.01 | 2.56 |
| 5′-O-phenylalanyl-Floxuridine | 1.58 ± 0.20* | 0.13 ± 0.01 | 12.34 |
| 3′,5′-di-O-valyl-Floxuridine | 2.78 ± 0.37* | 0.54 ± 0.01 | 5.18 |
| 3′-O-valyl-Floxuridine | 1.35 ± 0.13* | 1.32 ± 0.04 | 1.03 |
| 5′-O-valyl-Floxuridine | 3.42 ± 0.09* | 0.18 ± 0.01 | 19.24 |
| Floxuridine | Not detected | Not detected | — |
| 3′-O-L-valyl Gemcitabine | 1.12 ± 0.07 | 1.01 ± 0.03 | 1.11 |
| 5′-O-L-valyl Gemcitabine | 2.14 ± 0.05 | 0.18 ± 0.01 | 11.25 |
| 3′,5′-di-O-L-valyl Gemcitabine | 1.76 ± 0.09 | 1.52 ± 0.05 | 1.15 |
| 3′-O-D-valyl Gemcitabine | 0.81 ± 0.02 | 0.76 ± 0.03 | 1.06 |
| 5′-O-D-valyl Gemcitabine | 1.14 ± 0.04 | 0.72 ± 0.04 | 1.58 |
| 3′,5-di-O-D-valyl Gemcitabine | 1.11 ± 0.08 | 0.98 ± 0.06 | 1.13 |
| 3-O-L-isolecucyl Gemcitabine | 1.03 ± 0.11 | 0.94 ± 0.06 | 1.09 |
| 5′-O-L-isolecucyl Gemcitabine | 1.22 ± 0.05 | 0.21 ± 0.01 | 5.64 |
| 3′,5-di-O-L-isolecucyl Gemcitabine | 1.16 ± 0.13 | 1.09 ± 0.03 | 1.06 |
| 3′-O-L-phenylalanyl Gemcitabine | Not detected | Not detected | — |
| 5′-O-L-phenylalanyl Gemcitabine | Not detected | Not detected | — |
| 3′,5-di-O-L-phenylalanyl Gemcitabine | Not detected | Not detected | — |
| 3′-O-D-phenylalanyl Gemcitabine | Not detected | Not detected | — |
| 5′-O-D-phenylalanyl Gemcitabine | Not detected | Not detected | — |
| 3′,5′-di-O-D-phenylalanyl Gemcitabine | Not detected | Not detected | — |
| Gemcitabine | Not detected | Not detected | — |
| Valacyclovir | 2.51 ± 0.28 | 0.59 ± 0.06 | 4.25 |

### Example 7

Testing of Floxuridine Prodrugs for Activation with a Prototype Activation Enzyme—BPHL

To look at activation of the amino acid prodrug (e.g., the removal of the amino acid ester), the prodrugs are tested for hydrolysis using the prototype activation enzyme—purified biphenyl hydrolase-like enzyme (BPHL) (Kim I, Chu X Y, Kim S, Provoda C J, Lee K D, Amidon G L—Identification of a human valacyclovirase: biphenyl hydrolase-like protein as valacyclovir hydrolase. J Biol Chem. 2003 Jul. 11;278(28):25348-56). A solution containing 1 mM of each compound is incubated with the enzyme at 25° C. The reaction is stopped by the addition of 5% trifluoroacetic acid and the amount of parent compound is determined by HPLC analysis. Valacyclovir (VACV) hydrolysis by BPHL is used as a control. As seen in Table 5, the BPHL enzyme showed a range of hydrolytic activity that was dependent upon the linkage (5′ is favored over 3′) and on the identity of the promoiety (valyl>phenylalanine>lysine>aspartic acid).

TABLE 5
Activation of Floxuridine Prodrugs by purified BPHL.

| Floxuridine Prodrug | % of Control (VACV) |
|---|---|
| 3′,5′ valyl diester Floxuridine | 1.7% |
| 3′ valyl monoester Floxuridine | 3.3% |
| 5′ valyl monoester Floxuridine | 91.1% |
| 3′,5′ phenylalanyl diester Floxuridine | 8.9% |
| 3′ phenylalanyl monoester Floxuridine | 24.4% |
| 5′ phenylalanyl monoester Floxuridine | 52.8% |
| 3′,5′ aspartyl diester Floxuridine | 0.0% |
| 3′aspartyl monoester Floxuridine | 2.0% |
| 5′aspartyl monoester Floxuridine | 2.3% |
| 3′,5′ lysyl diester Floxuridine | 0.0% |
| 3′ lysyl monoester Floxuridine | 7.3% |
| 5′ lysyl monoester Floxuridine | 8.1% |
| Valacyclovir (VACV) | 100.0% |

### Example 8

Testing for Activation of Gemcitabine Prodrugs with Intestinal Cell Lysates and Plasma.

Confluent Caco-2 cells are washed with phosphate buffer saline (PBS, pH 7.4) and are harvested with 0.05% Trypsin-EDTA at 37° C. for 5-10 min. Trypsin was neutralized by adding DMEM. The cells are washed off the plate and spun down by centrifugation. The pelleted cells are washed twice with pH 7.4 phosphate buffer (10 mM), and resuspended in pH 7.4 phosphate buffer (10 mM) to obtain a final concentration of approximately 4.70×10⁶ cells/mL. The cells are lysed with one volume 0.5% Triton-X 100 solution. The cell lysate is homogenized by vigorous pipeting and total protein is quantified with the BioRad DC Protein Assay using bovine serum albumin as a standard. The hydrolysis reactions are carried out in 96-well plates (Corning, Corning, N.Y.). Caco-2 cell suspension (230 μl) is placed in triplicate wells and the reactions are started with the addition of substrate and incubated at 37° C. At various time points, 40 μL aliquots are removed and added to two volumes of 10% ice-cold TFA. The mixtures are centrifuged for 10 min at 1800 rcf and 4° C. and the supernatant filtered through a 0.45 μm filter. The recovered filtrate is analyzed by HPLC.

To test stability in human plasma, 230 μL undiluted plasma is added to each well in triplicate and 40 μL of substrate is added to start the reactions which are conducted at 37° C. for up to 4 hours. At various predetermined time points, 40 μL aliquots are removed and added to two volumes of 10% ice-cold TFA. The mixtures are centrifuged for 10 min at 1800 rcf at 4° C. and the supernatant is filtered through a 0.45 μm filter. The recovered filtrate is analyzed by HPLC.

The estimated half-lives (t_{1/2}), obtained from linear regression of pseudo-first-order plots of prodrug concentration vs time are listed in Table 6. The corresponding values for the two reference prodrugs, valacyclovir and valganciclovir are also listed in Table 6. The hydrolysis rates of the gemcitabine prodrugs and the reference prodrugs in plasma were significantly higher in plasma compared to that in phosphate buffer, pH 7.4. The hydrolysis rates of the prodrugs in Caco-2 cell homogenates are roughly comparable to that seen in plasma. These enhanced rates of degradation suggest specific enzymatic action. Two effects are noted: a) the effect of structure of promoiety on stability was in the order, isoleucyl>valyl>>phenylalanyl; and b) the stereochemistry of the promoiety affected the stability of the gemeitabine prodrugs in a profound manner (D-valyl and D-phenylalanyl prodrugs were roughly 4- to 14-fold more stable in Caco-2 cell homogenates than the corresponding L-analog).

TABLE 6
Activation of Gemcitabine prodrugs in buffer,
caco-2 cell homogenates and Human plasma.

| | t_{1/2} (min) | | |
|---|---|---|---|
| | | | Caco-2 cell |
| Prodrug | Buffer pH 7.4 | Human plasma | homogenates |
| 3′-O-L-valyl gemcitabine | 64.0 ± 1.4 | 5.4 ± 0.1 | 5.0 ± 0.1 |
| 5′-O-L-valyl gemcitabine | 416.0 ± 8.5 | 56.4 ± 2.9 | 7.1 ± 0.6 |
| 3′,5′-di-O-L-valyl gemcitabine | 55.0 ± 2.7 | 2.0 ± 0.1 | 0.9 ± 0.0 |
| 3′-O-D-valyl gemcitabine | 74.0 ± 1.2 | 5.99 ± 0.0 | 23.2 ± 0.2 |
| 5′-O-D-valyl gemcitabine | 424.0 ± 1.2 | 58.1 ± 2.1 | 37.4 ± 1.4 |
| 3′,5′-di-O-D-valyl gemcitabine | 52.0 ± 1.1 | 2.1 ± 0.1 | 10.3 ± 0.7 |
| 3′-O-L-isoleucyl gemcitabine | 66.0 ± 0.21 | 8.0 ± 0.1 | 10.6 ± 0.3 |
| 5′-O-L-isoleucyl gemcitabine | 452.0 ± 9.6 | 99.2 ± 1.6 | 75.3 ± 2.8 |
| 3′,5′-di-O-L-isoleucyl gemcitabine | 61.0 ± 0.5 | 2.64 ± 0.0 | 2.1 ± 0.0 |
| 3-O-L-phenylalanyl gemcitabine | 39.0 ± 0.12 | 5.7 ± 0.1 | 0.8 ± 0.0 |
| 5′-O-L-phenylalanyl gemcitabine | 200.0 ± 1.9 | 8.4 ± 0.2 | 3.2 ± 0.1 |
| 3′,5′-di-O-L-phenylalanyl gemcitabine | 38.0 ± 0.2 | 0.7 ± 0.0 | 0.6 ± 0.0 |
| 3′-O-D-phenylalanyl gemcitabine | 39.0 ± 0.7 | 7.7 ± 0.2 | 8.8 ± 0.1 |
| 5′-O-D-phenylalanyl gemcitabine | 204.0 ± 3.5 | 34.8 ± 1.1 | 11.4 ± 0.2 |
| 3′,5′-di-O-D-phenylalanyl gemcitabine | 28.0 ± 0.6 | 2.4 ± 0.2 | 8.3 ± 0.6 |
| Valacyclovir | 1029.0 ± 11.4 | 312.0 ± 24.6 | 27.7 ± 0.6 |
| Valganciclovir | 990.0 ± 14.4 | 303.0 ± 18.0 | 32.7 ± 0.7 |

### Example 9

Synthesis of Amino Acid Prodrugs of Ara A that Contain no Nucleophile on the Amino Acid Side Chain.

Abbreviations used herein: Ara A is Adenine 9-beta-D-arabinofuranoside, DCC is N,N′-Dicyclohexylcarbodiimide, DMF is dimethylformamide, equ is equivalents, HPLC is High Performance Liquid Chromatography, NMR is Nuclear Magnetic Resonance, and TFA is Trifluoroacetic acid, DCM is dichloromethane, DMAP is dimethyl amine pyridine, TBAF is Tetrabutylammonium fluoride, t-Boc is tertiary-butyloxycarbonyl, and t-butyl is tertiary butyl.

As shown in Scheme 3, DCC (1.5 equ) in DMF solution is added to an anhydrous DMF solution containing Ara A (1 equ), dried N Boc protected Amino acid (1.5 equ) and dried dimethyl amine pyridine (1.5 equ). The mixture is stirred at room temperature for 20 hours, during which time a white precipitate forms. At the end of this period, 5 mL of anhydrous ethanol is added and the reaction mixture is stirred for an additional 1 hour. The solution is filtered to remove the precipitate and the filtrate is evaporated to dryness. The residue is subjected to flash silica gel chromatography to separate the 2′ and 3′ monoester and diester prodrugs from the 5′ monoester prodrug. The protected prodrug is collected and dried by vacuum. The residue is deprotected by 50% TFA in dichloromethane and purified by preparative HPLC. All of the prodrug compounds, which are made by this method and are identified in Table 5, are characterized by H-NMR, C13-NMR, Mass Spectrometry, and elemental analysis. The purities of the compounds are verified by HPLC and elemental analysis and are above 97% purity. All of the compounds are isolated as TFA salts and are very soluble in water.

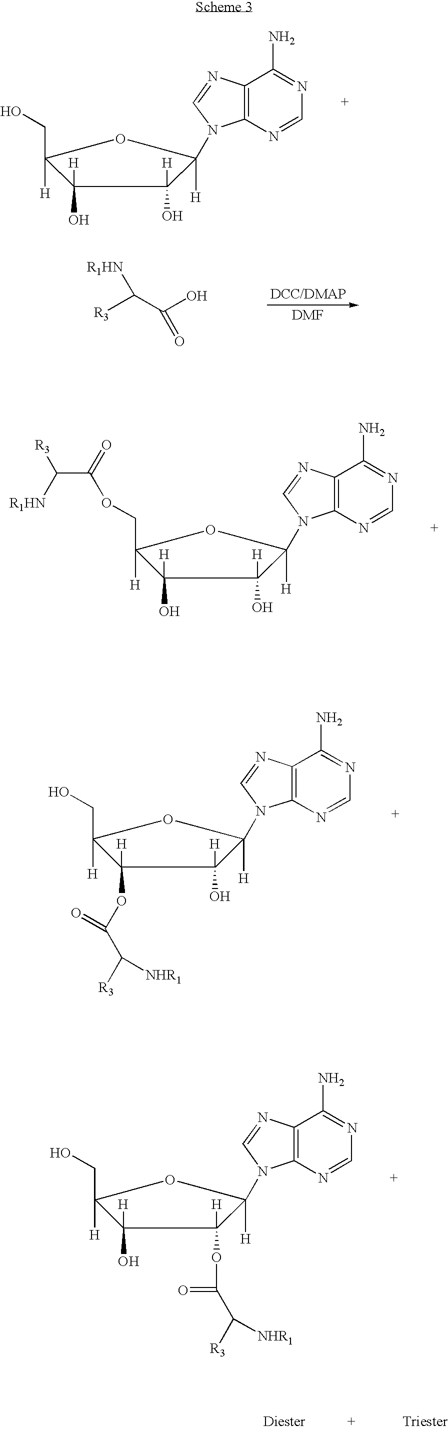

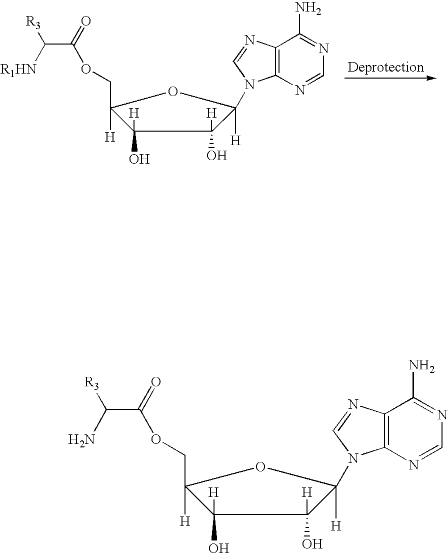

Scheme 3 shows synthesis of Ara A amino acid prodrugs that do not contain a nucleophile in the amino acid side chain. In this scheme R₁ represents a protective group such as t-Boc that is commonly used to protect amine groups and R₃ is the side chain of the amino acid used in the synthesis of the prodrug.

### Example 10

Synthesis of the Amino Acid Prodrugs of Ara A with Amino Acids Containing a Nucleophilic Side Chain,

For this method, the 2′ and 3′ hydroxyl groups of Ara A are protected to prevent formation of the 2′ and 3′ mono and diester prodrugs, see Scheme 4. Tert-butyldimethylchlorosilane (1.2 equ) in 20 ml DMF is added to a solution of dried 9-(beta-D-arabinofuranosyl)adenine (1 equ) and imidazole (3 equ) in 10 mL of dry N,-N-dimethylformamide (DMF) and the mixture is stirred under argon at room temperature for 24 h. The solvent is removed in vacuo at 42° C. and the residue is purified by silica flash chromatography with a solvent of 92:8 dichloromethane and methanol. The product, 9-[5-O-(tert-Butyldimethylsilyl)-beta-D-arabinofuranosyl]adenine(16), is crystallized from a mixture of hot chloroform and hexane with a yield of 90%.

DCC (9 equ) is added to a solution of laevulin acid (9 equ) in dry ethyl acetate (25 mL) and the mixture is stirred under the protection of argon room temperature for 1.5 h. The mixture is filtered under argon and added to a suspension of 16 (1 equ) and N,N-dimethylaminopyridine (1.2 equ) in dry ethyl acetate (25 mL). The reaction mixture is stirred at room temperature for 4 h, dry EtOH (10 mL) is added and stirred an additional 30 min, and the reaction is filtered. The filtrate is extracted three times with 50 mL of ice-cold saturated NaHCO solution and the combined aqueous solution is back washed three times with 50 mL ethyl acetate. The combined ethyl acetate solution is extracted with ice-cold water (3×50 mL) and the combined water layer is back washed three times with ethyl acetate. The washed ethyl acetate solution is evaporated to dryness. The residue is subjected to flash column silica gel chromatography developed with dichloromethane followed by 94:6 dichloromethane and methanol. The product—9-[5′-O-(tert-Butyldimethylsilyl)-2′,3′, dilevulinyl-beta-D-arabinofuranosyl] adenine (17) is isolated at a yield of 100%.

The syrup of 17 (1 equ) is dissolved in a solution of 1 M TBAF in THF (5 ml) containing glacial acetic acid (0.5 mL). The reaction mixture is stirred at room temperature for 2 h and is then evaporated to dryness in vacuo in the presence of toluene. The residue is separated by silica gel flash chromatography developed with 95:5 and 92:8 dichloromethane and methanol, yielding a clear gum, which is further separated by silica gel flash chromatography developed with 9:1 dichloromethane and methanol. The dried product 9-[2′,3′-dilevulinyl-l-beta-D-arabinofuranosyl] adenine (18) is isolated with 70% yield.

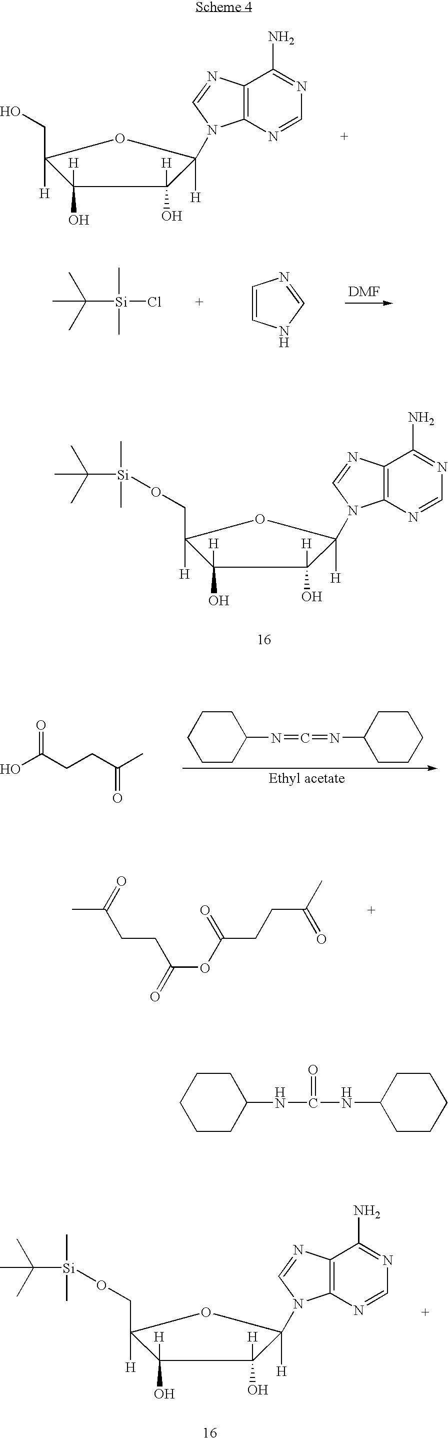

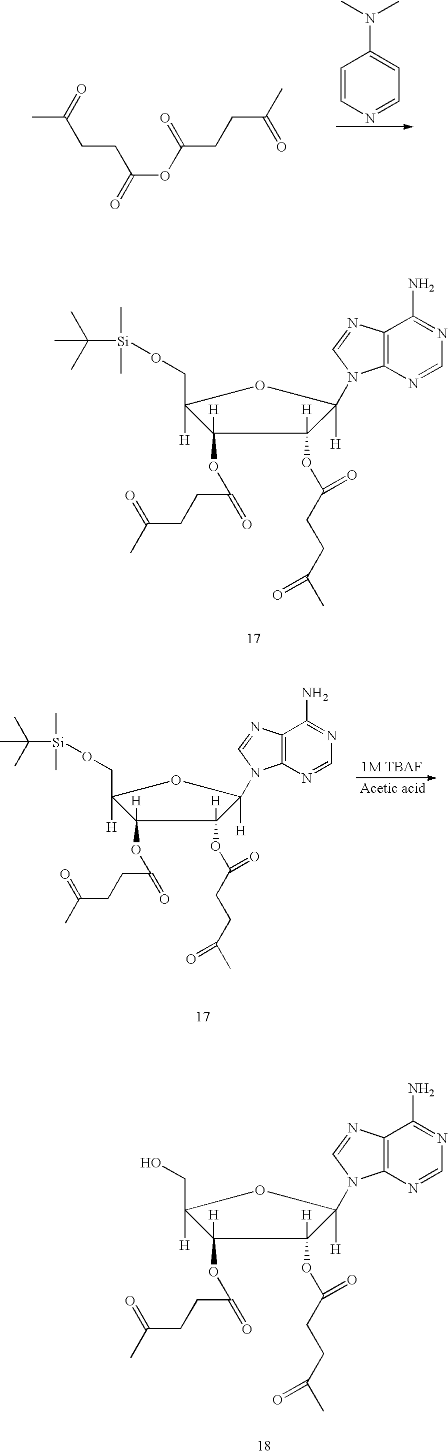

Scheme 4 shows the synthesis of 9-[2′,3′-dilevulinyl-l-beta-D-arabinofuranosyl] adenine (18), an intermediate for the synthesis of 5′ O amino acid ester prodrugs of Ara A in which the amino acid has a reactive side chain group.

The synthesis of 5′ O-ester Ara A prodrugs is shown in Scheme 5. DCC solution (1.5 equ) in DMF is added over a time course of 10 minutes to a DMF solution containing the protected Ara A 18 (1 equ), N protected Amino acid (1.5 equ) and Dimethyl amine pyridine (1.5 equ). The mixture is stirred at room temperature for 20 hours during which time a white precipitate formed. At the end of this period, 5 mL of anhydrous ethanol is added and the reaction mixture is stirred for an additional 1 hour. The solution is filtered to remove the precipitate and the filtrate is evaporated to dryness. The residue, containing the protected Ara A prodrug (19) is dissolved in ethyl acetate, and consecutively washed with saturated ammonium chloride aqueous solution (1 time) and water (1 time). The organic phase is dried in vacuo and the residue is subjected to flash silica gel chromatography. The product, 19, is dissolved in 4:1 pyridine and glacial acetic acid followed by addition of 0.1M of hydrazine monohydrate in 4:1 pyridine and glacial acetic acid to remove the levulinic groups from the 2′ and 3′ OH groups of the Ara A. The deprotected prodrug, 20, is separated by flash silica gel chromatography and the amino acid protection group(s) are removed either by TFA (t-Boc, t-butyl) or by hydrogenolysis (Z or benzyl groups) or both and the final product, 21, is purified by preparative HPLC. All of the compounds are identified by H-NMR, C13-NMR, Mass Spectrometry, and elemental analysis. The purities of the compounds should be verified by HPLC and elemental analysis and are above 97% purity. All of the compounds are isolated as TFA salts and are very soluble in water.

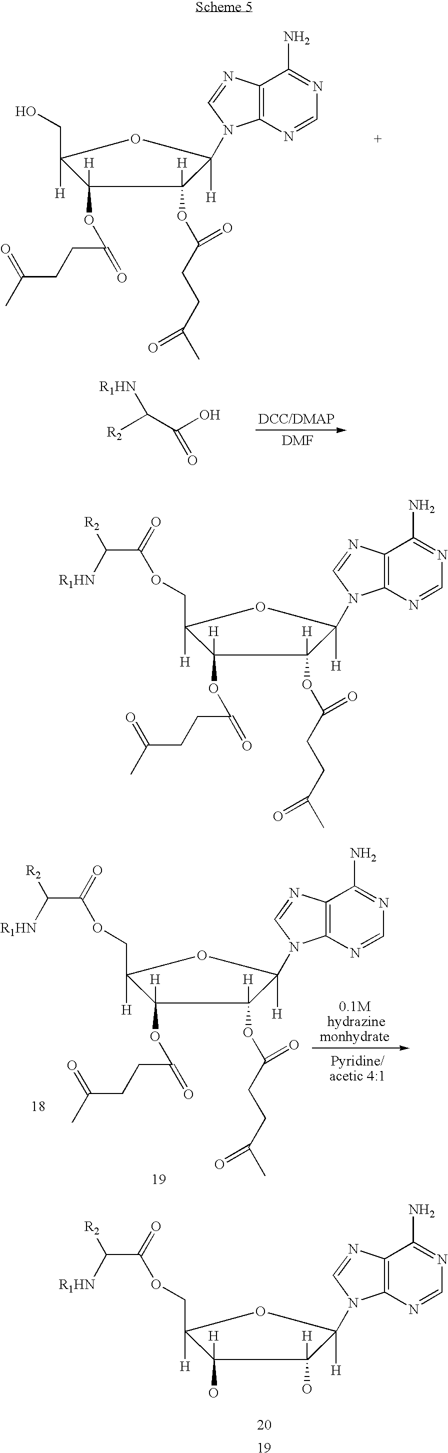

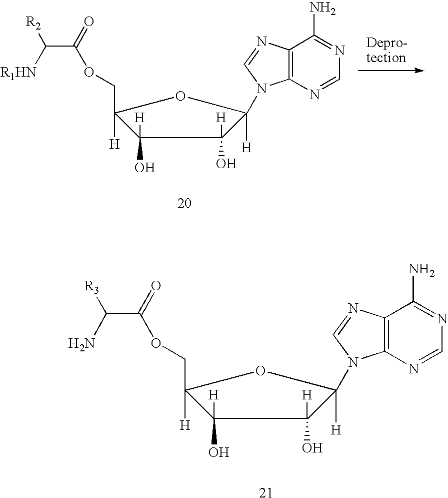

Scheme 5 shows synthesis of 5′ O-ester Ara A prodrugs (21) using an amino acid that has a reactive side chain group. Such amino acids illustratively include lysine, serine, cysteine, glutamine, asparagine, threonine, tyrosine and an amino acid having the formula HOOC—(CH₂)ₙ—CH(NH₂)—COOH where n is an integer in the range of 1-6, inclusive. In this scheme R₁ represents a protective group such as t-Boc that is commonly used to protect amine groups, R₂ represents a protected side chain of the amino acid that is used in the synthesis of the prodrug and R₃ is the deprotected side chain of the amino acid used in the synthesis of the prodrug.

### Example 11

Synthesis of 5′ O-ester Ara A Prodrugs Including an Amino Acid Containing a Reactive Carboxylic Group of the Side Chain.

In particular embodiments, an amino acid conjugated to Ara A has the formula HOOC—(CH₂)ₙ—CH(NH₂)—COOH where n is an integer in the range of 1-6, inclusive.

Scheme 6 illustrates synthesis of 5′ O-ester Ara A prodrugs (26) in which the reactive carboxylic group of the side chain of the amino acid, aspartic acid (n=1), glutamic acid (n=2) or analogs thereof (n=3-6), is linked to the 5′ hydroxyl group of Ara A.

Protection of 2′ and 3′ OH groups of Ara A is performed as described in Example 10 and Scheme 4. The synthesis of 5′ O-ester Ara A prodrugs (Scheme 6) is as described in Example 10, but uses a starting protected amino acid 22 that is blocked at the alpha carboxylic group and the alpha amino group and with a carboxylic group on the side chain of the amino acid.

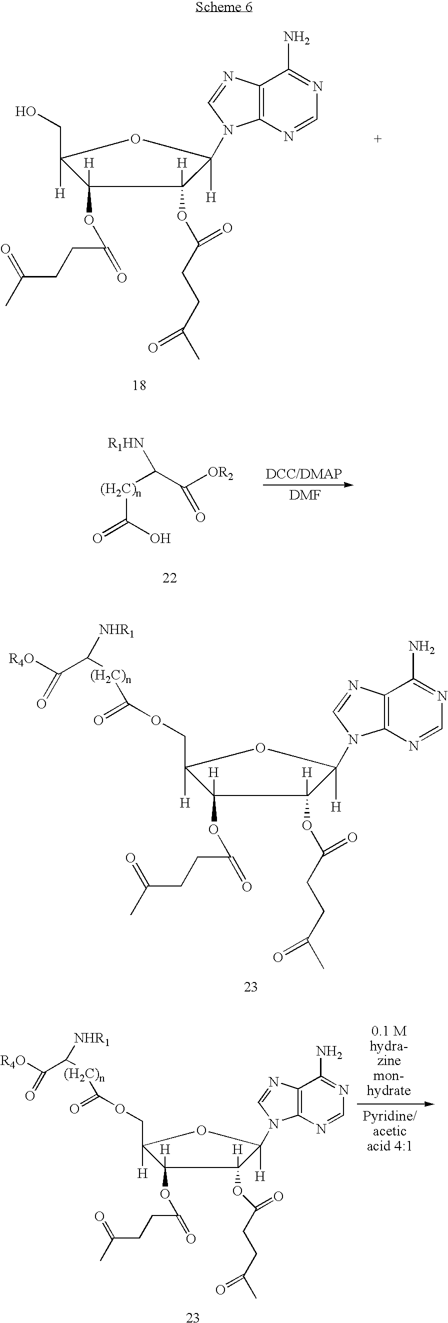

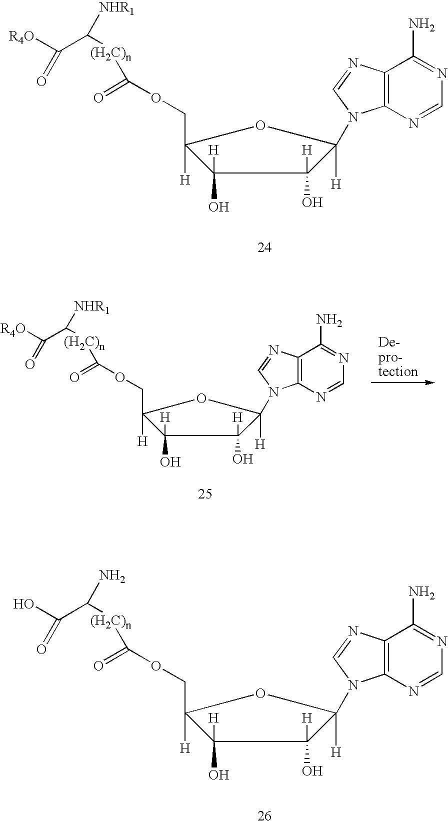

Scheme 6 shows synthesis of 5′ O-ester Ara A prodrugs (26) in which the reactive carboxylic group of the side chain of amino acids aspartic acid (n=1), glutamic acid (n=2) and their analogs (n=3-6) is linked to the 5′ hydroxyl group of Ara A. In this scheme R₁ represents an amine protective group such as t-Boc commonly used Lo protect amine groups and R₄ is a protective group, such as a benzyl group, commonly used to protect carboxylic groups.

TABLE 7
Examples of Amino Acids Prodrugs of Ara A that were synthesized
by “Method 1”, “Method 2” or “Method 3” described
in Examples 9, 10 and 11, respectively.

| | Amino Acid | Molecular | Estimated | Synthetic |
|---|---|---|---|---|
| Prodrug Designation | Promoiety | Weight | Log P¹ | Method |
| 5′-O-D-isoleucyl Ara A | D-ILE | 380.4 | −0.85 | Method 1 |
| 5′-O-L-isoleucyl Ara A | L-ILE | 380.4 | −0.85 | Method 1 |
| 5′-O-D-valyl Ara A | D-VAL | 366.3 | −1.27 | Method 1 |
| 5′-O-L-valyl Ara A | L-VAL | 366.3 | −1.27 | Method 1 |
| 5′-O-glycyl Ara A | GLY | 324.2 | −2.65 | Method 1 |
| 5′-O-D-phenylalanyl Ara A | D-PHE | 414.4 | −0.48 | Method 1 |
| 5′-O-L-phenylalanyl Ara A | L-PHE | 414.4 | −0.48 | Method 1 |
| 5′-O-D-leucyl Ara A | D-LEU | 380.4 | −0.92 | Method 1 |
| 5′-O-L-leucyl Ara A | L-LEU | 380.4 | −0.92 | Method 1 |
| 5′-O-D-alpha-aspartyl Ara A | D-alpha ASP | 382.33 | −2.94 | Method 2 |
| 5′-O-L-alpha-aspartyl Ara A | L-alpha ASP | 382.33 | −2.94 | Method 2 |
| 5′-O-D-beta-aspartyl Ara A | D-beta ASP | 382.33 | −2.94 | Method 3 |
| 5′-O-L-beta-aspartyl Ara A | L-beta ASP | 382.33 | −2.94 | Method 3 |

¹Estimated from Pro Log P program used in ChemDraw Pro, version 9.

### Example 12

Antiviral Activity of Ara A and Its Prodrugs

Antiviral activity of inventive prodrugs is determined using standard antiviral assays.

For example, the anti-pox virus activity of Ara A and its prodrugs is determined using standard vaccinia virus assays. Virus (˜50 plaques per well in 6-well cluster plates containing confluent HFF cells) is overlayed with a methocel drug mixture (at drug concentrations ranging from 0.03 to 100 micromolar) and is incubated for 3 days. Plaques are visualized with 0.1% crystal violet in 20% methanol. Drug effects are calculated as a percentage of the reduction in plaque number in the presence of each drug concentration compared to the numbers obtained in the absence of drug. The 50% inhibitory IC₅₀ values are calculated using standard methods. As seen in Table 8, Ara A has significant antipox virus activity with IC₅₀ values that are 4-5 times lower than cidofovir, the positive control for this virus. Co-administration of the Ara A with the adenosine deaminase inhibitor 2′-deoxycoformycin (at a concentration of 1 micromolar) gives IC₅₀ values that are 5 (vaccinia) to 10 (cowpox) fold lower than those seen with Ara A alone. At this low level (1 micromolar), the 2′-deoxycoformycin alone has no effect on virus replication; however, with a K₁ of ˜50 nM as an ADA inhibitor, its effect is likely the result of inhibition of adenosine deaminase. The amino acid ester prodrugs of Ara A show IC₅₀ values comparable to Ara A alone and there is little observed difference between D and L amino acid prodrugs with regard to antipox or cytotoxicity.

TABLE 8
Antiviral and cytotoxicity data for prodrugs Ara A

| | Vaccinia | Cowpox | |
|---|---|---|---|
| | Plaque | Plaque | KB cell |
| | Reduction | Reduction | cytotoxicity |
| | (IC₅₀- | (IC₅₀- | (IC₅₀- |
| Compounds | micromolar) | micromolar) | micromolar) |
| Ara A | 5.0 | 6.5 | >100 |
| Ara A + 2′- | <<1.0 | 0.65 | 90 |
| deoxycoformycin¹ | | | |
| 5′-O-L-phenylalanyl Ara A | 20 | nd | >100 |
| 5′-O-L-valyl Ara A | 12 | nd | 65 |
| 5′-O-L-isoleucyl Ara A | 4.5 | nd | >100 |
| 5′-O-L-prolyl Ara A | 12 | nd | 45 |
| 5′-O-L-aspartyl Ara A | 7.0 | nd | 60 |
| 5′-O-Glycyl Ara A | 15 | nd | >100 |
| 5′-O-D-valyl Ara A | 3.5 | nd | >100 |
| 5′-O-D-isoleucyl Ara A | 3.0 | nd | >100 |
| Ara-H | nd | >100 | nd |
| Cidofovir | 26 | 30 | >100 |

¹2-deoxycoformycin was co-incubated with the Ara A at a concentration of 1 micromolar.
The abbreviation “nd” in Table 8 stands for “not done.”

### Example 13

Solution and Biological Homogenate Stability of 5′-O-amino acid prodrugs of Ara A.

The chemical stabilities of the Ara A prodrugs are determined in pH 7.4 phosphate buffer (10 mM) at 37° C. in order to obtain the contribution of non-enzymatic hydrolysis. The hydrolysis reactions are carried out in 96-well plates. PBS buffer (230 microliters) is placed in triplicate wells and the reactions are started with the addition of Ara A prodrugs and incubated at 37° C. At various time points, 40 microliter aliquots are removed and added to 40 microliter of 10% ice-cold TFA. The mixtures are centrifuged for 10 min at 2000×g at 4° C. then filtered through a 0.45 micron filter. The filtrate is analyzed by HPLC. The prodrugs are stable at low pH (data not shown), and the Phe, Val, and Ile ester prodrugs of Ara A show reasonable chemical stability at physiological pH, with mean t_{1/2} times ranging from 379 to 425 minutes (Table 9). The aspartyl prodrug has reduced stability, relative to the aforementioned ester prodrugs.

Stability of the Ara A prodrugs in Caco-2 homogenates is illustrated in this example. Homogenates are prepared by adding an equal volume of 0.5% Triton-X 100 to 5×10⁶ Caco-2 cells/mL followed by vigorous vortexing. The hydrolysis experiments are carried out in triplicate as described above in chemical stability section using cell homogenate instead of buffer. As can be seen in Table 9, the hydrolysis of all prodrugs to Ara A is significantly faster in the Caco-2 homogenates than in buffer alone, indicating enzymatic conversion of the prodrug to Ara A. In Minimal Essential Medium with Earle's salts (MEME) buffer, which is used in the vaccinia and cowpox plaque reduction assays described in Example 12, the isoleucyl prodrug shows the greatest stability.

TABLE 9
Estimated half-life of Ara A prodrugs in pH 7.4 phosphate
buffer, Caco-2 cell homogenates, and MEME (n = 3).

| | t_{1/2} (min) | | |
|---|---|---|---|
| Prodrugs | pH 7.4 | Caco-2 | MEME |
| 5′-O-L- | 420.9 ± 40.1 | 3.5 ± 1.0 | ND |
| phenylalanyl | | | |
| Ara A | | | |
| 5′-O-L- | 425.25 ± 50.1 | 11.1 ± 0.4 | ND |
| valyl Ara A | | | |
| 5′-O-L- | 378.0 ± 17.9 | 27.0 ± 1.3 | 137 ± 12.5 |
| isoleucyl Ara A | | | |
| 5′-O-L- | 20.1 ± 1.0 | 1.3 ± 0.005 | 14.8 ± 0.6 |
| prolyl Ara A | | | |
| 5′-O-L- | 141.4 ± 26.1 | ND | ND |
| aspartyl Ara A | | | |
| Valacyclovir | 990 ± 14.4 | 32.7 ± 0.7 | ND |

ND = not done

### Example 14

Stability Studies of 5′-O-valyl and 5′-O-isoleucyl Ara A Prodrugs in the Presence of Adenosine Deaminase.

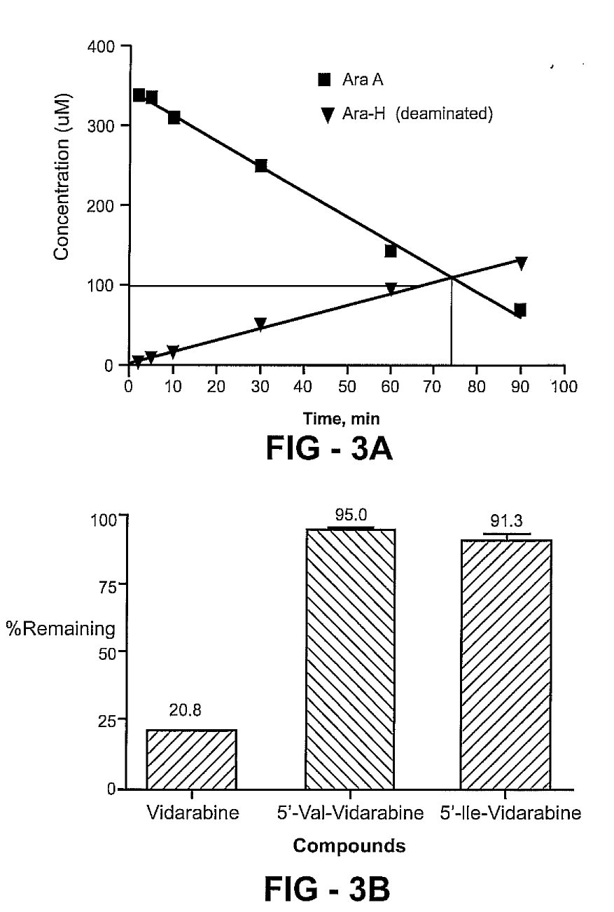

Another aspect of the prodrug strategy for Ara A is to minimize the activity of adenosine deaminase (ADA) activity that converts the Ara A to 9-(beta-D-arabinofuranosyl) hypoxanthine (Ara-H), which has less anti-pox virus activity than Ara A. Significant levels of this enzyme are found in the intestinal mucosa, which limits the effectiveness of oral Ara A delivery. To test for stability of Ara A and the representative prodrugs 5-O-L-Valine Ara A and 5-O-L-Isoleucine Ara A in the presence of adenosine deaminase, 230 microliters of a 0.2 mg/mL stock solution of adenosine deaminase is placed in wells of 96-well plate, the reactions are initiated by the addition of the prodrug substrates (200 micromolar) in pH 7.4 phosphate buffer, and are incubated at 37° C. for 90 minutes. A minimum of three wells is used for each compound tested. At various time points, 40 microliter aliquots are removed and added to 40 microliters 10% cold TFA. The mixtures are centrifuged and filtered through a 0.45 micron filter for 10 min at 2000×g and 4° C. The filtrate is then analyzed by HPLC. The disappearance of Ara A or prodrug as well as the appearance of the deaminated product is monitored to determine stability profiles. FIGS. 3A and 3B show deamination of Ara A, 5-O-L-Valine Ara A and 5-O-L-Isoleucine Ara A adenosine deaminase. In particular, FIG. 3A shows concentration (micromolar) profiles of the disappearance of Ara A and the appearance of Ara-H in the presence of adenosine deaminase. This figure shows that Ara A is rapidly hydrolyzed with a commensurate rise in the concentration of the deaminated product, ara-H. FIG. 3B shows the percent Ara A or prodrug remaining following incubation with adenosine deaminase for 90 minutes.

This figure also shows that the 5′-O-amino acid prodrugs of Ara A are resistant to the enzyme, with >90% remaining after a 90-minute incubation with excess enzyme. These results indicate that the prodrugs were capable of withstanding the metabolic effects of the ADA.

### Example 15

Cellular Uptake Studies of Ara A Prodrugs:

Another aspect of the prodrug strategy for Ara A is to improve the absorption of the drug through the intestinal tract. In particular, amino acid ester prodrugs of Ara A are designed to target transport of the prodrug to be mediated by nutrient transporters, e.g., the dipeptide transporter PepT1, that reside in the intestinal membrane. In these investigations, the test system for carrier-mediated uptake is HeLa cells that are transiently transfected with the human dipeptide transporter, hPept1. For the transient transfection, HeLa cells were seeded into multiwell plates at a density of 1×10⁵ cells/cm² and incubated for 24 hours. The cells are transfected with an expression plasmid containing the hPept1 gene under the control of the CMV promoter and are used for uptake studies within 48 hours of the transfection. A 1 mM prodrug solution in phosphate transport buffer, pH=6.0 is incubated for 45 minutes with HeLa/hPept1 cells two days post-transfection. The cells are lysed with a 1:1 mixture of MeOH and water, then centrifuged (20 min at 12,000×g), filtered and analyzed by HPLC. HeLa cells are used as a negative control. Protein amount of each sample is determined with the Bio-Rad Protein Assay using bovine serum albumin as a standard. Data are reported in Table 10 as nmole transported/mg of protein/45 minutes. The fold enhancement of the carrier mediated uptake is calculated from the HeLa cell control. The 5-O′-L-Valyl- and 5-O′-L-isoleucyl-Ara A prodrugs show 20.7 and 26.9 fold transport enhancement, respectively, which is unexpectedly greater than that seen with valacyclovir, a known prodrug of acyclovir described in Han, H., et al., Pharmaceutical Research, 1998. 15(8):1154-1159.

TABLE 10
Direct uptake of the monopeptide L-Valyl, L-Isoleucyl,
L-prolyl, L-aspartyl, and the dipeptide L-Ala-Glycyl
Ara A prodrugs in HeLa/hPEPT1 and HeLa cells.

| | HeLa/hPEPT1 | HeLa control | |
|---|---|---|---|
| | (nmol/mg/ | (nmol/mg/ | hPEPT1/ |
| Prodrugs | 45 min) | 45 min) | Control |
| 5′-O-L- | 80.3 ± 1.1 | 3.9 ± 0.3 | 20.7 |
| valyl Ara A | | | |
| 5′-O-L- | 202.0 ± 4.8 | 7.5 ± 0.2 | 26.9 |
| isoleucyl Ara A | | | |
| 5′-O-L- | 40.8 ± 1.0 | 38.5 ± 1.3 | 1.06 |
| prolyl Ara A | | | |
| 5′-O-L- | 5.0 ± 0.2 | 3.9 ± 0.6 | 1.27 |
| aspartyl Ara A | | | |
| 5′-O-L- | 10.9 ± 0.8 | 7.4 ± 0.3 | 1.48 |
| alagly Ara A | | | |
| Valacyclovir | 221.2 ± 8.1 | 11.6 ± 0.5 | 19.0 |

### Example 16

Caco-2 Monolayer Transport and Stability Studies Using Ara A and the Prodrugs 5′-O-L-valyl Ara A, 5′-O-L-isoleucyl Ara A.

The transepithelial transport of Ara A, 5′-O-L-valyl Ara A, 5′-O-L-isoleucyl Ara A is tested in Caco-2 monolayers grown in transwell filters following standard procedures as described in detail in Landowski, C. P. et al., Pharm Res, 2005. 22(9): p. 1510-8. In these experiments, transport studies are performed 21 days post-seeding. The assay is initiated by adding drug transport solution (0.2 mM drug in MES buffer, pH 6.0 containing 5 mM D-glucose, 5 mM MES, 1 mM CaCl2, 1 mM MgCl₂, 150 mM NaCl, 3 mM KCl, 1 mM NaH2PO4) to the apical chamber of the Caco-2 insert. Two hundred microliter aliquots are withdrawn from the basolateral chamber at predetermined intervals and replaced with fresh HEPES pH 7.4 buffer. The epithelial integrity of representative cell monolayers is assessed by monitoring transepithelial resistance. In these assays, the stability of the drug in both chambers of the insert is also monitored. As seen in Table 11, the permeabilities of the two prodrugs are enhanced by 3 to 4 fold. Importantly, the prodrugs are stable, showing minimal metabolism to Ara A and Ara-H over a 2-hour time frame.

TABLE 11
Transport and stability of Ara A and select prodrugs
in Caco-2 cell monolayer (n = 3)

| | | Stability¹ (% remaining) | |
|---|---|---|---|
| | | Apical | Basolateral |
| | Permeability | chamber | chamber |
| Prodrugs | (×10⁻⁶, cm/s) | (donor) | (receiver) |
| Ara A | 1.42 ± 0.05 | — | — |
| 5′-O-L-valyl Ara A | 3.86 ± 0.03 | 82.1 | 62.0 |
| 5′-O-L-isoleucyl Ara A | 5.66 ± 0.14 | 89.6 | 77.5 |

¹Percent prodrug remaining in donor or receiver solution at 120 minutes

### Example 17

Intestinal Absorption Studies of Ara A and Selected 5′-O-amino Acid Prodrugs of Ara A.

The absorption of selected amino acid prodrugs of Ara A is illustrative of the advantage of the prodrug approach to development of an oral dosage product of Ara A. In order to minimize complications of gastric dilution or degradation, intestinal absorption is measured by injection of a prodrug or drug solution directly into the duodenal intestinal segment of an anesthetized rat. The systemic plasma profiles of the prodrug, Ara A, and its main metabolite, Ara H are then monitored by removing plasma from a jugular vein cannula over a 4 hour period and assaying by LC/MS/MS assay.

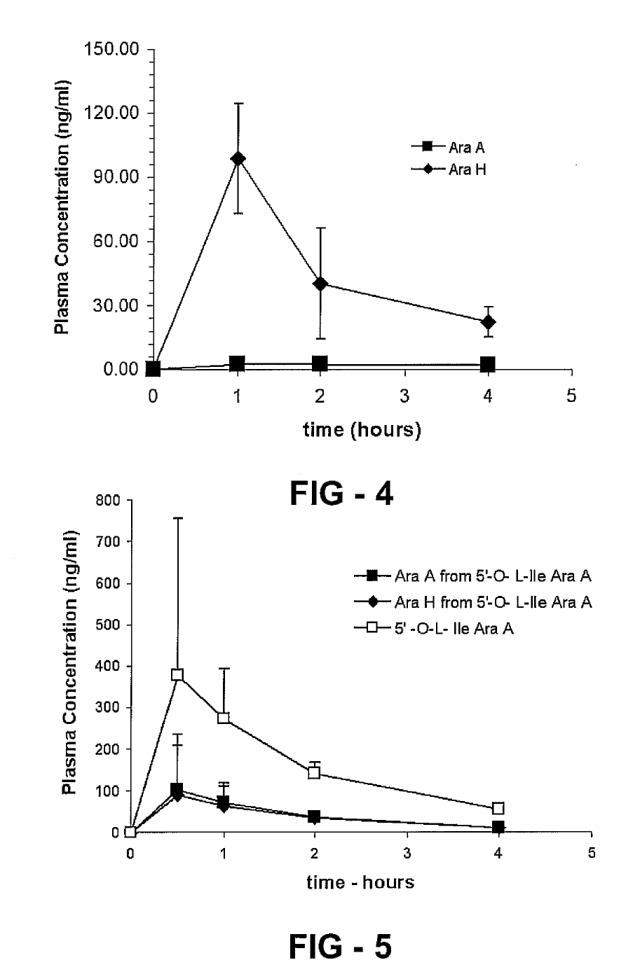

FIG. 4 shows a graph illustrating the plasma profiles of Ara A and Ara H after administration of Ara A into the duodenum of a rat. Animals were dosed with Ara A at a level of 1.5 mg/rat via direct duodenal injection into anesthetized rats (n=2 per compound). Plasma samples were taken from the jugular vein at 0, 1, 2, and 4 hours from the jugular vein and were assayed for Ara A (▪), and Ara H (♦) by LC/MS/MS. For Ara A dosing, over 95% of the drug is converted to ara-H as exemplified in FIG. 4. However, for the prodrugs, significant levels of Ara A and the prodrugs themselves are detected.

FIG. 5 shows a graph of mean plasma profiles of Ara A, Ara H, and 5′-O-L isoleucyl Ara A after administration of 5′-O-L isoleucyl Ara A into the duodenum of a rat. Animals were dosed with 5′-O-L isoleucyl Ara A prodrug at a level of 2.7 mg/rat via direct duodenal injection into anesthetized rats (n=3 per compound). Plasma samples were taken from the jugular vein at 0, 0.5, 1, 2, and 4 hours from the jugular vein and were assayed for prodrug (□), Ara A (▪), and Ara H (♦) by LC/MS/MS. FIG. 5 illustrates that in the case of 5′-O-L-Ile Ara A approximately 17% of the total drug measured in plasma is present as Ara A, 15% of the total drug measured is found to be Ara-H, and 68% of the total drug measured is present as Ile-Ara A, based on AUC0-4 hr calculations, as shown in Table 12. The 5′-O-L-Ile Ara A prodrug results in >20 fold increase in circulating Ara A levels over dosing with Ara A.

TABLE 12
AUC0-4 hr values for Ara A and Ile-Ara A.

| | AUC0-4 hr values (ng/mL · hr) | | |
|---|---|---|---|
| Dosing Drug or Prodrug | Ara A | Ara-H | Prodrug- |
| Ara A | 8.3 | 181.5 | — |
| 5′-O-L-Ile-Ara A | 172 +/− 107 | 156 ± 110 | 658 ± 314 |
| 5′-O-L-Val-Ara A | 126 ± 1.4 | 145 ± 21 | 195 ± 25 |
| 5′-O-D-Val-Ara A | 109 ± 14 | 98 ± 16 | 637 ± 74 |

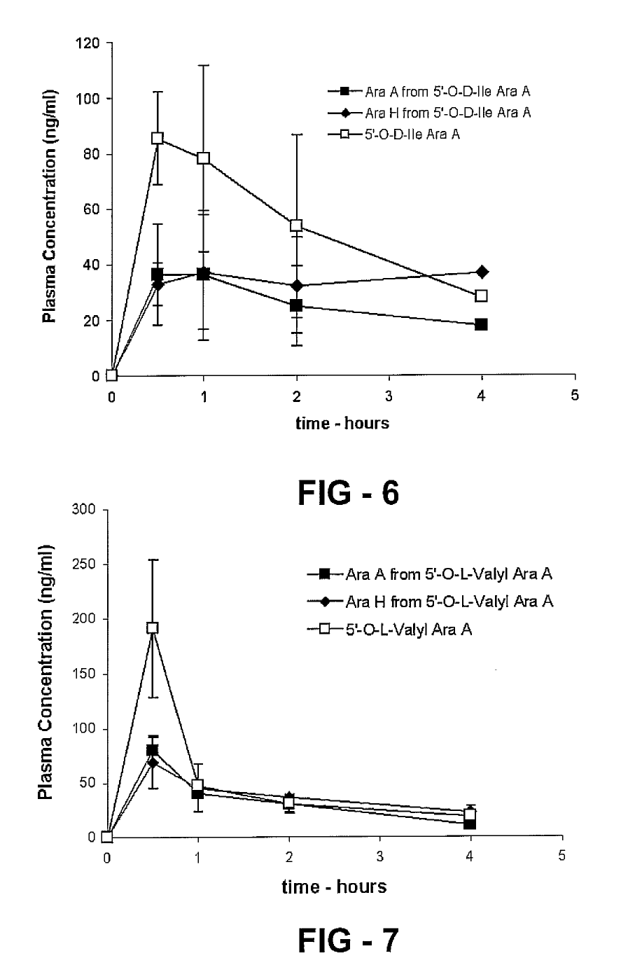

FIG. 6 is a graph showing mean plasma profiles of Ara A, Ara H, and 5′-O-D Isoleucyl Ara A after administration of 5′-O-D Isoleucyl Ara A into the duodenum of a rat. Animals were dosed with 5′-O-D Isoleucyl Ara A prodrug at a level of 2.7 mg/rat via direct duodenal injection into anesthetized rats (n=2). Plasma samples were taken from the jugular vein at 0, 0.5, 1, 2, and 4 hours from the jugular vein and were assayed for prodrug (□), Ara A (▪), and Ara H (♦) by LC/MS/MS. As shown in FIG. 6, increased levels of plasma Ara A are found after dosing with the D-5′-O-Ile Ara A prodrug.

FIG. 7 is a graph showing mean plasma profiles of Ara A, Ara H, and 5′-O-L Valyl Ara A after administration of 5′-O-L Valyl Ara A into the duodenum of a rat. Animals were dosed with 5′-O-L Valyl Ara A prodrug at a level of 3 mg/rat via direct duodenal injection into anesthetized rats (n=2). Plasma samples were taken from the jugular vein at 0, 0.5, 1, 2, and 4 hours from the jugular vein and were assayed for prodrug (□), Ara A (▪), and Ara H (♦) by LC/MS/MS.

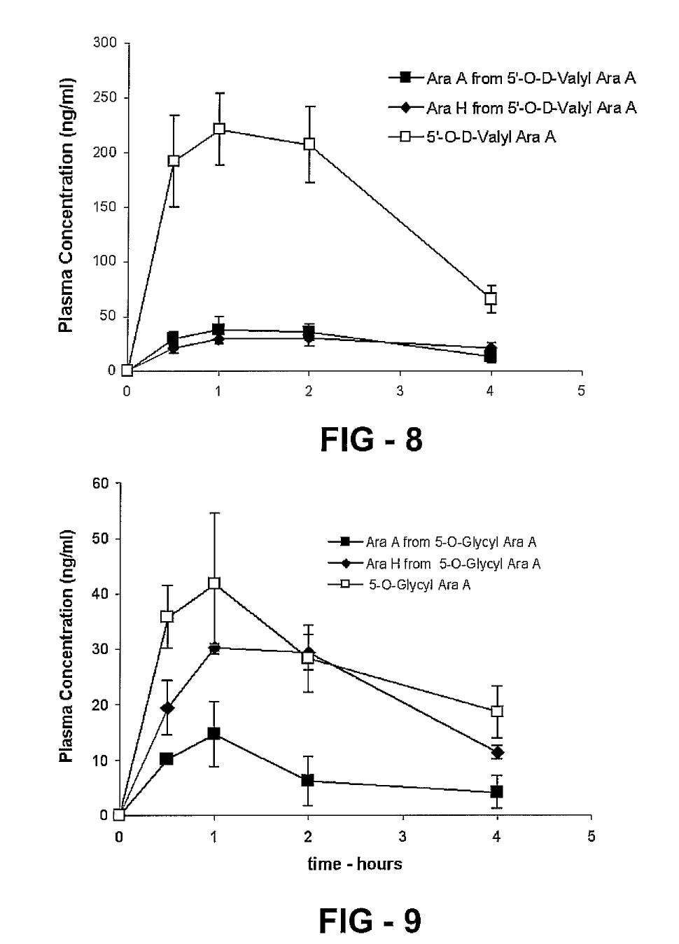

FIG. 8 is a graph showing mean plasma profiles of Ara A, Ara H, and 5′-O-D Valyl Ara A after administration of 5′-O-D Valyl Ara A into the duodenum of a rat. Animals were dosed with 5′-O-D Valyl Ara A prodrug at a level of 3.0 mg/rat via direct duodenal injection into anesthetized rats (n=3). Plasma samples were taken from the jugular vein at 0, 0.5, 1, 2, and 4 hours from the jugular vein and were assayed for prodrug (□), Ara A (▪), and ara H (♦) by LC/MS/MS.

Increased levels of plasma Ara A are found after dosing with the L- and D-5′-O-valyl prodrugs as demonstrated in FIGS. 7 and 8. There is a marked difference in the plasma profiles of the D-valyl Ara A prodrug over that seen with its L-stereoisomer that may have a important ramifications in the further development of the prodrugs. It is seen that the D-valyl prodrug shows extended and enhanced concentrations of the prodrug in systemic plasma. These data suggest that the D-isomer may be able to act as a circulating reservoir of Ara A, in the form of the D-amino acid prodrug.

FIG. 9 is a graph showing mean plasma profiles of Ara A, Ara H, and 5′-O-Glycyl Ara A after administration of 5′-O-Glycyl Ara A into the duodenum of a rat. Animals were dosed with 5′-O-Glycyl Ara A prodrug at a level of 3.0 mg/rat via direct duodenal injection into anesthetized rats (n=2). Plasma samples were taken from the jugular vein at 0, 0.5, 1, 2, and 4 hours from the jugular vein and were assayed for prodrug (□), Ara A (▪), and Ara H (♦) by LC/MS/MS. For the glycyl prodrug, there was limited intestinal absorption of the prodrug and relatively low plasma levels of the prodrug, Ara A and Ara H, likely due to instability of the prodrug.

### Example 18

Solubility Testing of Ara A Prodrugs

Water is added dropwise to 5 mg of each prodrug with intermittent stirring until the drug is fully dissolved. All of the Ara A prodrugs dissolved prior to the addition of 0.19 ml of water or at a concentration>26 mg/ml water. In contrast, unmodified Ara A is poorly soluble (<0.5 mg/ml) under these conditions.

Any patents or publications mentioned in this specification are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The apparatus and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A composition, comprising:
a prodrug having the structural formula: 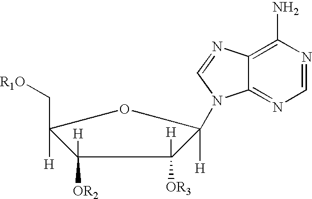
where R₁, R₂ and R₃ are each independently H, or a substrate for a transporter selected from the group consisting of: an amino acid, a dipeptide and a tripeptide, where at least one of R₁, R₂ and R₃ is an amino acid, a dipeptide or a tripeptide; or a salt or hydrate thereof.

2. The composition of claim 1, wherein the amino acid is an L-amino acid.

3. The composition of claim 1, wherein the amino acid is a D-amino acid.

4. The composition of claim 1, where R₁ is selected from the group consisting of: an amino acid, a dipeptide and a tripeptide, and where R₂ and R₃ are each independently H, an amino acid, a dipeptide or a tripeptide.

5. The composition of claim 1, where R₁ is selected from the group consisting of: an amino acid, a dipeptide and a tripeptide, and where R₂ and R₃ are both H.

6. The composition of claim 1, wherein the transporter is an intestinal transporter.

7. The composition of claim 1, further comprising an inhibitor of adenosine deaminase.

8. The composition of claim 1, characterized by at least two-fold greater bioavailability compared to adenine 9-beta-D-arabinofuranoside.

9. A composition, comprising:
a prodrug having the structural formula 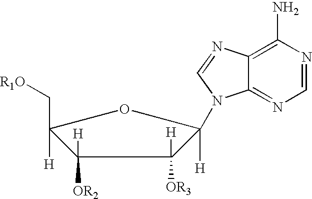
where R₁ is an amino acid and where R₂ and R₃ are both H.

10. The composition of claim 9, wherein the amino acid is a non-polar amino acid.

11. The composition of claim 9, wherein the amino acid is a substrate for an intestinal transporter.

12. A composition of claim 9, wherein the amino acid is a D-amino acid.

13. The composition of claim 9, wherein the amino acid has the formula HOOC—(CH₂)ₙ—CH(NH₂)—COOH where n is an integer in the range of 1-6, inclusive.

14. The composition of claim 9, wherein the prodrug is selected from the group consisting of: 5′-O-D-isoleucyl Ara A; 5 ′-O-L-isoleucyl Ara A; 5′-O-D-valyl Ara A; 5 ′-O-L-valyl Ara A; 5′-O-glycyl Ara A; 5′-O-D-phenylalanyl Ara A; 5′-O-L-phenylalanyl Ara A; 5′-O-D-leucyl Ara A; 5′-O-L-leucyl Ara A; 5′-O-L-aspartyl Ara A; 5′-O-D-alpha-aspartyl Ara A; 5′-O-L-alpha-aspartyl Ara A; 5′-O-D-beta-aspartyl Ara A; 5′-O-L-beta-aspartyl Ara A; and 5′-O-L-prolyl Ara A.

15. A method of treatment of a subject, comprising:
administering to a subject in need thereof a therapeutically effective amount of a composition comprising a prodrug having the structural formula: 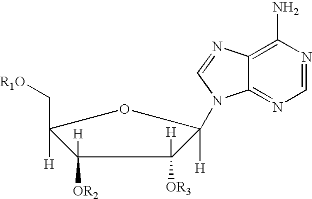
where R₁, R₂ and R₃ are each independently H, an amino acid, a dipeptide or a tripeptide, where at least one of R₁, R₂ and R₃ is an amino acid, a dipeptide or a tripeptide; and a pharmaceutically acceptable carrier.

16. The method of claim 15, wherein the subject is infected or at risk of infection with a virus from a virus family selected from the group consisting of: Adenoviridae, Arenaviridae, Bunyaviridae, Caliciviridae, Coronaviridae, Flaviviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, and Togaviridae.

17. The method of claim 16, wherein the subject is human.

18. The method of claim 15, wherein the administration is oral administration.

19. The method of claim 15, wherein the subject has or is at risk of having cancer.
